(19) United States
(12) Patent Application Publication
(10) Pub. No.: US 2018/0055777 A1
(43) Pub. Date: Mar. 1, 2018

(54) Hydrogel Beads With Self-Regulating Microclimate pH Properties

(71) Applicant: UNIVERSITY OF MASSACHUSETTS, BOSTON, MA (US)

(72) Inventors: David Julian McClements, Northampton, MA (US); Zipei Zhang, Amherst, MA (US); Ruojie Zhang, Amherst, MA (US)

(21) Appl. No.: 15/690,748

(22) Filed: Aug. 30, 2017

Related U.S. Application Data
(60) Provisional application No. 62/381,748, filed on Aug. 31, 2016.
(60) Provisional application No. 62/402,017, filed on Sep. 30, 2016.

(51) Int. Cl. A61K 9/48; A61K 9/00; A61K 9/50; A61K 38/46; A61K 38/47; B01J 13/14; B01J 13/00; A23L 33/10; A23L 33/00

(52) U.S. Cl. CPC A61K 9/485; A61K 9/0053; A61K 9/5089; A61K 38/465; A61K 38/47; A23V 2002/00; B01J 13/14; B01J 13/0065; A23L 33/10; A23L 33/30; A61K 9/5036

(57) ABSTRACT

A composition includes a hydrogel bead in an external matrix, the hydrogel bead having an at least partially crosslinked gelling polymer, the hydrogel bead having encapsulated therein a functional agent, and a buffering agent having low water solubility, wherein at least a portion of the buffering agent is in solid form in the hydrogel bead.

### CROSS-REFERENCE TO RELATED APPLICATIONS

This application claims priority to U.S. Provisional Application Ser. No. 62/381,748 filed on Aug. 31, 2016, and U.S. Provisional Application Ser. No. 62/402,017 filed on Sep. 30, 2016, which are incorporated herein by reference in their entirety.

### STATEMENT REGARDING FEDERALLY SPONSORED RESEARCH & DEVELOPMENT

This invention was made with government support under 2014-67017-21635 and 2016-31100-06025 awarded by the USDA, National Institute of Food and Agriculture (NIFA). The government has certain rights in the invention.

### FIELD OF THE DISCLOSURE

The present disclosure is related to hydrogel beads, specifically hydrogel beads for delivery of bioactive agents.

### BACKGROUND

There is considerable interest in the development of colloidal delivery systems to encapsulate, protect and deliver bioactive agents (nutrients, micronutrients, nutraceuticals, and drugs) in the food, supplement, and pharmaceutical industries. Numerous types of bioactive agents can be encapsulated and delivered such as proteins, peptides, amino acids, probiotics, prebiotics, vitamins, minerals, nutraceuticals, triacylglycerol oils, flavors, colors, antimicrobials, preservatives, and the like. For example, bioactive proteins are often encapsulated due to their sensitivity to chemical degradation and their potential for causing off-flavors (such as bitterness or astringency). Probiotic bacteria can be protected from acidic stomach conditions as they move through the gastrointestinal tract (GIT) to areas where their beneficial actions are most effective (such as the colon). Many lipophilic bioactive agents (e.g., curcumin and carotenoids) are encapsulated to address the limitations of their incorporation into food products such as low water-solubility, poor chemical instability, and low bioaccessibility. Many natural colors and flavors are encapsulated to inhibit their chemical degradation under unfavorable environmental conditions.

Research has indicated that encapsulation in colloidal delivery systems can improve the handling, applicability, chemical stability, and oral bioavailability of many bioactive agents. Colloidal delivery systems may be fabricated using different procedures and methods based on the molecular and physicochemical properties of the bioactive agents. One of the most promising approaches to create colloidal delivery systems is to fabricate hydrogel beads with bioactive agents trapped inside. Hydrogel beads normally contain hydrophilic, porous, and elastic polymer matrices, which can improve the stability of bioactive agents during storage and control their release after oral intake. Because hydrogel beads are formed using mild preparation conditions with food-grade biopolymers (e.g., polysaccharides and protein) that do not usually adversely affect the properties of encapsulated bioactive agents, they can be found in the applications of a broad range fields from food to medicine.

Many bioactive agents are highly susceptible to physical, chemical or biological degradation under one set of pH conditions, but are relatively stable under another set of pH conditions. For example, some natural colors rapidly fade under highly acidic pH conditions, but slowly under neutral conditions. Many probiotics (bacteria beneficial to gut health) are rapidly inactivated under highly acidic conditions, but remain viable under neutral conditions. A major drawback associated with the use of conventional hydrogel beads for the encapsulation of these pH-labile bioactive agents is that they are highly porous, and so acid or alkaline determining ions (such as H⁺ or OH⁻) can easily diffuse in or out of them, thereby causing the pH inside the beads to be fairly similar to that outside. As a result, any pH-labile bioactive agents may be readily degraded when they are encapsulated in this type of bead. The pH sensitivity of conventional hydrogel beads therefore hinders their widespread applications in the food, supplement, and pharmaceutical industries for encapsulating pH-labile bioactive agents.

Research has been carried out to improve the pH-stability of substances encapsulated in hydrogel beads, e.g., by coating the beads with one or more layers of biopolymers using electrostatic deposition, or by crosslinking the hydrogel matrix using specific enzymes. These approaches are based on delaying the diffusion of pH determining ions into the hydrogel beads. However, these approaches have been shown to have a limited protective effect and also have other limitations, i.e., the fabrication procedure is complex and the encapsulated bioactive agents are not released at the desired site of action.

What is needed are new strategies for providing hydrogel beads that can improve the stability of encapsulated bioactive agents.

### BRIEF SUMMARY

In one aspect, a composition comprises a hydrogel bead in an external matrix, the hydrogel bead comprising an at least partially crosslinked gelling polymer, the hydrogel bead having encapsulated therein a functional agent, and a buffering agent having low water solubility, wherein at least a portion of the buffering agent is in solid form in the hydrogel bead.

In another aspect, also included are pharmaceutical and food compositions comprising the hydrogel beads.

In yet another aspect, a method of making an encapsulated functional agent comprises mixing a gelling polymer, a functional agent, and a buffering agent having low water solubility to form a mixture, forming hydrogel beads from the mixture, and crosslinking at least a portion of the gelling polymer in the hydrogel beads to provide the encapsulated functional agent, wherein at least a portion of the buffering agent is in solid form in the hydrogel bead.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram showing the fabrication of self-adjusting microclimate pH hydrogel beads; the beads are formed by injecting alginate, bioactive, and insoluble buffer into a cross-linking solution.

FIG. 2 is a schematic representation of a hydrogel bead of the present disclosure.

FIG. 3 shows fluorescent images of phosphate buffer at pH 2.5, 3, 4, 5, 6, and 7, respectively. The images of the first row (TMR channel) and second row (FITC channel) were emitted at 543 and 488, detect at 650/LP and 590/50 respectively.

FIG. 4 shows a standard curve of pH vs. intensity ratio taken by microplate at concentrations of 2.5, 5 and 10 μL/ml stock solution (10 mg/mL).

FIG. 5 shows a standard curve of pH vs. intensity ratio taken by confocal microscope at 5 μL/ml stock solution (10 mg/mL).

FIG. 6 shows differential interference contrast (DIC) and fluorescent images of hydrogel beads without Mg(OH)₂ encapsulation before going through the stomach phase.

FIG. 7 shows differential interference contrast (DIC) and fluorescent images of hydrogel beads with 0.3% Mg(OH)₂ encapsulation before going through stomach phase. For FIGS. 6 and 7, the images of the TMR channel and FITC channel were emitted at 543 and 488, detected at 650/LP and 590/50 respectively. The images of the ratio channel were generated by using image J software and the color strip represents the pseudo-color change with pH on the right top (intensity of TMR signal was enhanced using Image J to improve contrast).

FIG. 8 shows an illustration of the image processing undertaken to generate a gradient pH profile of hydrogel beads without Mg(OH)₂ encapsulation. The ratio image is presented with pseudocolor color (Fire).

FIG. 9 shows the same pixel thickness rings as FIG. 8 obtained from the bead outline.

FIG. 10 shows the pH value of each averaged ring as a function of distance from the bead diameter for buffer free beads.

FIG. 11 shows an illustration of the image processing undertaken to generate a gradient pH profile of hydrogel beads with Mg(OH)₂ encapsulation. The ratio image is presented with pseudocolor color (Fire).

FIG. 12 shows the same pixel thickness rings as FIG. 11 obtained from the bead outline.

FIG. 13 shows the pH value of each averaged ring as a function of distance from the bead diameter for buffer loaded beads.

FIG. 14 shows the fluorescent images of hydrogel beads without Mg(OH)₂ encapsulation after going through stomach phase (intensity of TMR signal was enhanced using Image J to improve contrast).

FIG. 15 shows the fluorescent images of hydrogel bead with 0.3% Mg(OH)₂ encapsulation after going through stomach phase. In FIGS. 14 and 15, the images of the first row (TMR channel) and second row (FITC channel) were emitted at 543 and 488, detect at 650/LP and 590/50 respectively. The images of the ratio channel were generated by using image J software and the color strip represents the pseudocolor change with pH on the right top (intensity of TMR signal was enhanced using Image J to improve contrast).

FIG. 16 shows differential interference contrast (DIC) images of hydrogel beads with Mg(OH)₂ encapsulation taken during 2 h stomach phase incubation.

FIG. 17 is graphical representation of the pH value of hydrogel beads without and with Mg(OH)₂ (0.3%) encapsulation during 2 h stomach digestion process.

FIG. 18 shows the predicted time dependence of the fraction of hydrogen ions (H₃O+) absorbed by alginate beads with a diameter of 200 μm and a pore size of 10 nm after submersion in an acidic environment.

FIG. 19 shows a standard curve of pH vs. intensity ratio taken by 5 μL/ml stock solution (10 mg/mL).

FIG. 20 shows fluorescent images of hydrogel beads without buffer encapsulation during the digestion process. The images of the TMR channel and FITC channel were emitted at 543 and 488, detect at 650/LP and 590/50 respectively (intensity of TMR signal was enhanced using Image J to improve contrast).

FIG. 21 shows fluorescent images of hydrogel beads with buffer encapsulation during the digestion process. The images of the TMR channel and FITC channel were emitted at 543 and 488, detect at 650/LP and 590/50 respectively (intensity of TMR signal was enhanced using Image J to improve contrast).

FIG. 22 shows the amount of free fatty acids released from hydrogel bead with or without buffer encapsulation using a pH-stat in vitro digestion model.

FIG. 23 shows confocal microscope of lipase-loaded beads with/without buffer encapsulation after intestine digestion. Lipase was dyed with FITC to show green color while the lipid was dyed by Nile Red to show red color.

FIG. 24 shows particle size distributions of different samples initially and after the stomach digestion process.

FIG. 25 shows mean particle diameter (d₄₃) of different samples initially and after the stomach digestion process.

FIG. 26 shows the relative activity of β-galactosidase encapsulated in large hydrogel beads after incubation in simulated gastric fluids for different times. The enzyme was encapsulated in hydrogel beads containing different amounts of Mg(OH)₂ (0-0.30%). Samples designated with different letters (a, b) were significantly different (Duncan,p<0.05) when compared between different delivery systems for the final point (120 min).

FIG. 27 shows the relative activity of β-galactosidase encapsulated in small hydrogel beads after incubation in simulated gastric fluids for different times. The enzyme was encapsulated in hydrogel beads combined with different amounts of Mg(OH)₂ (0-0.80%). Samples designated with different letters (a, b, c) were significantly different (Duncan, p<0.05) when compared between different delivery systems for the final point (120 min).

FIG. 28 shows the relative activity of β-galactosidase encapsulated in hydrogel beads after 120 min of incubation in simulated gastric fluids. The enzyme was encapsulated in hydrogel beads containing different amounts of Mg(OH)₂.

FIG. 29 shows the visual appearance of the color change of lactase-loaded large beads incubated in the o-NPG solution at pH 2.5 without buffer co-encapsulation.

FIG. 30 shows visual appearance of the color change of the o-NPG solution at pH 7 without buffer co-encapsulation.

FIG. 31 shows the visual appearance of the color change of the o-NPG solution at pH 2.5 with 0.3% Mg(OH)₂ co-encapsulation.

FIG. 32 is a schematic representation of physicochemical processes occurring in the lactase-loaded beads incubated in o-NPG solution at pH 2.5 with Mg(OH)₂ co-encapsulation.

FIG. 33 shows particle size distributions of carrageenan beads fabricated by syringe (large beads) and Encapsulator (small beads).

FIG. 34 shows visual appearances of carrageenan beads fabricated by syringe (large beads) and Encapsulator (small beads).

FIG. 35 shows the predicted time dependence of the fraction of hydrogen ions (H3O [symbol] ) absorbed by carrageenan beads with a diameter of 255 μm (small size beads) and 2609 μm (larger size beads) with a pore size of 15 nm after submersion in an acidic environment.

The above-described and other features will be appreciated and understood by those skilled in the art from the following detailed description, drawings, and appended claims.

### DETAILED DESCRIPTION

Described herein are hydrogel beads with adjustable internal pH microclimates. The hydrogel beads include a buffering agent having low water solubility, which maintains the pH inside of the hydrogel beads. Because the buffering agent has low water solubility, at least a portion of the buffering agent is in solid form in the hydrogel bead. Low solubility, as used herein, refers to a water solubility of less than 2×10⁻² g per 100 g of water.

Specifically, a composition comprises a hydrogel bead in an external matrix. Exemplary external matrices include aqueous matrices, air, an organic solvent, or an oil. A specific external matrix is an aqueous matrix. The hydrogel bead comprises an at least partially crosslinked gelling polymer and has encapsulated therein a functional agent, and a buffering agent having low water solubility. At least a portion of the buffering agent is in solid form in the hydrogel bead.

In an aspect, the composition comprises 0.1 wt % to 70 wt % of the gelling polymer, 0.001 wt % to 60 wt % of the functional agent, and 0.001 wt % to 10 wt % of the buffering agent, wherein all weights are based on the total mass of the hydrogel beads.

The hydrogel beads may have an internal pH which is either higher, similar, or lower than the external pH. In an aspect, the pH in the hydrogel bead is higher or lower than the pH of the external matrix. In a specific aspect, the hydrogel beads have a pH that is higher than that of the surrounding matrix, which we refer to as antacid hydrogel beads.

The hydrogel beads comprise an at least partially crosslinked gelling polymer. Exemplary gelling polymers comprise a polysaccharide, a glycoprotein, a glycopeptide, a protein carbohydrate conjugate, a protein lipid conjugate, a carbohydrate-lipid conjugate, a protein, or a combination comprising at least one of the foregoing. Specific gelling polymers comprise alginate, carrageenan, agar, pectin, xanthan gum, chitosan, whey protein, caseinate, soy protein, pea protein, legume protein, gelatin, lactoferrin, or a combination comprising at least one of the foregoing. It is preferred that the at least partially crosslinked gelling polymer comprises sufficient crosslinking to provide a three-dimensional network that fills the hydrogel bead.

Alginate, for example, is a food-grade ingredient already widely utilized for encapsulating bioactive agents (e.g., proteins, enzymes, lipids, nutraceuticals). Besides the mild cross-linking process, the acid insolubility of alginate gels makes them a good candidate for encapsulation of bioactive agents to enhance stability in strong acid conditions (e.g., the stomach).

The hydrogel beads also include a buffering agent having low water solubility, for example, Ca(OH)₂, CaCO₃, Ca₃(PO₄)₂, Mg(OH)₂, MgCO₃, Zn(OH)₂, ZnCO₃, Zn₃(PO₄)₂, or a combination comprising at least one of the foregoing. The water solubility of

TABLE 1
The solubility and basicity of exemplary basic salts

| | | Water Solubility |
|---|---|---|
| Salts | pKₛₚₐ | g per 100 g of water |
| Ca(OH)₂ | 5.26 | 1.11 × 10⁻² |
| CaCO₃ | 8.42 | 6.17 × 10⁻⁵ |
| Ca₃(PO₄)₂ | 26.0 | 3.12 × 10⁻¹¹ |
| Mg(OH)₂ | 10.74 | 1.66 × 10⁻⁴ |
| MgCO₃ | 5.00 | 3.16 × 10⁻³ |
| Zn(OH)₂ | 15.68 | 3.74 × 10⁻⁶ |
| ZnCO₃ | 10.78 | 4.07 × 10⁻⁶ |
| Zn₃(PO₄)₂ | 32.0 | 1.24 × 10⁻¹³ |

Exemplary acidic buffers include organic acids such as benzoic acid, salicylic acid, and mineral acids such as metasilicic acid.

Preferably, the buffering agents should have a very low water solubility (water solubility <2×10⁻² g per 100 g of water). The water-solubility can be determined by measuring the amount dissolved in an aqueous solution in equilibrium with the crystalline form.

The hydrogel beads include a functional agent such as a bioactive agent, a colorant, a flavoring, a nutrient, a supplement, or a combination comprising one or more of the foregoing. The term supplement includes a nutrient or a non-nutrient chemical. A supplement is normally needed due to the insufficiency of the specific ingredient in human body, such as an enzyme. A nutrient, in contrast, may be always needed by human body, such as vitamin A, or D.

The hydrogel beads also include a bioactive agent such as a protein, an enzyme, a nutraceutical, a probiotic, a prebiotic, a drug or a combination comprising at least one of the foregoing. Exemplary bioactive agents include β-galactosidase, lipase, lactase, phospholipase, amylase, pepsin, chymotrypsin, trypsin, a carotenoids (α-carotene, β-carotene, lycopene, lutein, and fucoxanthin), curcumin, resveratrol, a polyphenol, a phytosterol, a phytostannol, a flavonoid, Coenzyme Q10, or a combination comprising at least one of the foregoing.

In an aspect, the hydrogel beads have an average diameter of 500 nm to 5000 μm, specifically 160 μm to 5000 μm, and more specifically 300 μm to 600 μm.

In an aspect, the composition is in the form of an oral pharmaceutical or neutraceutical composition comprising a pharmaceutically acceptable carrier. A method of administering a bioactive agent comprises orally administering a composition as disclosed herein to a subject, such as a human subject. In an aspect, upon orally administering, the buffering agent having low water solubility maintains a basic pH in the hydrogel beads through the subject's stomach.

Pharmaceutically acceptable excipients include diluents, preservatives, solubilizers, emulsifiers, and adjuvants. As used herein “pharmaceutically acceptable excipients” are well known to those skilled in the art.

Tablets and capsules for oral administration may be in unit dose form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavoring or coloring agents.

In another aspect, the composition is in the form of a food composition. The term “basal food composition” refers to a food composition combinable with additives such as the peptides and antibodies described herein. Basal animal food compositions may include components such as proteins, grains, flavor compositions, vitamins, minerals, preservatives, and the like. Basal food compositions can be suitable for ingestion by a target animal.

The beads are particularly suitable for utilization within the food, supplement, and pharmaceutical industries because of their simplicity of fabrication, low-cost, effectiveness, and the fact that they can be produced using edible ingredients (proteins and polysaccharides). The hydrogel beads can be used to incorporate acid-sensitive natural colors, flavors, or nutraceuticals into acidic food products such as beverage, juices, sauces, dressings and soups. Alternatively, it could also be used in the food, supplement and pharmaceutical industries to improve the stability of acid-sensitive bioactives (such as proteins, enzymes, nutraceuticals, probiotics, and drugs) in the gastrointestinal tract of humans or animals.

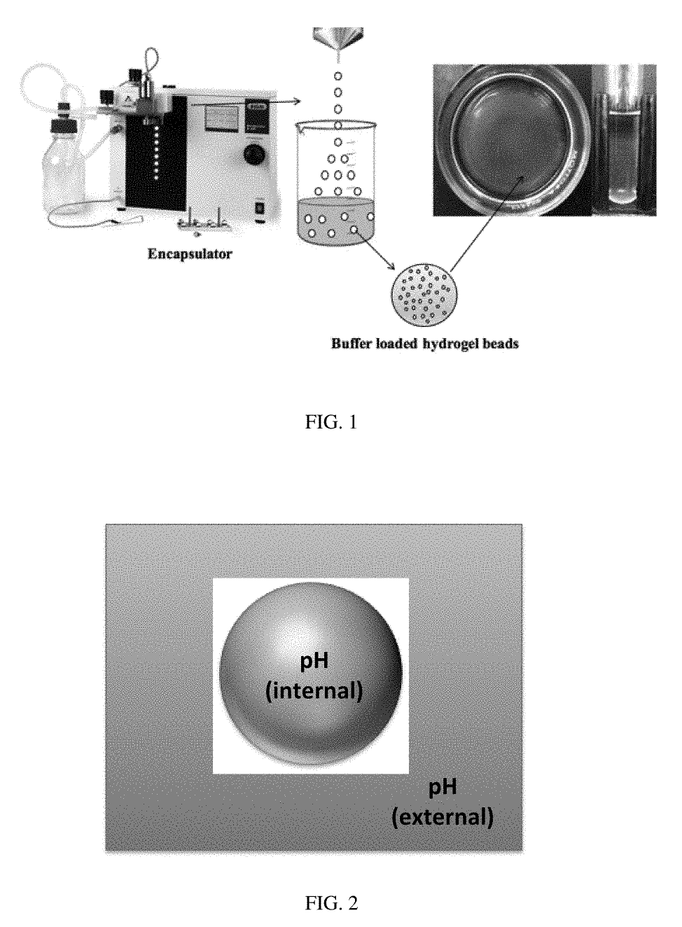

Hydrogel beads having diameters of greater than 1 mm to 5 mm, in one embodiment, are prepared using a syringe with a needle or a pipette. Smaller hydrogel beads, for example, having diameters of 160 μm to 1 mm, can be prepared using a specialized extrusion device (Encapsulator) with a vibrating nozzle (FIG. 1). The properties of the hydrogel beads (dimensions, rigidity and stability) formed using this method can be adjusted by altering several factors including the gelling polymer, the cross-linker concentration, instrument parameter such as nozzle type, frequency, electrode, operating pressure, and crosslinking time.

In an embodiment, a method of making an encapsulated functional agent comprises mixing a gelling polymer, a functional agent, and a buffering agent having low water solubility to form a mixture, forming hydrogel beads from the mixture, and crosslinking at least a portion of the gelling polymer in the hydrogel beads to provide the encapsulated functional agent, wherein at least a portion of the buffering agent is in solid form in the hydrogel bead.

In an embodiment, forming hydrogel beads from the mixture comprises passing the mixture through a vibrating nozzle, or through a needle. Hydrogel beads may also be formed using a variety of other methods, such as complex coacervation, thermodynamic incompatibility, or antisolvent precipitation methods.

Crosslinking at least a portion of the gelling polymer can comprise incubating the hydrogel beads in a solution comprising a cationic crosslinking agent for a time sufficient to crosslink at least a portion of the gelling polymer. Exemplary cationic crosslinking agents include Ca²⁺, K⁺, Na⁺, chitosan, polylysine, or a combination comprising at least one of the foregoing.

The composition of the beads can be selected to control their behavior within the gastrointestinal tract. In particular, the beads should remain intact within the product, mouth, and stomach, and should retain the bioactive component in these environments. However, the beads should disintegrate and/or release the bioactive components under small intestine or colonic conditions (depending on the application). The type of gelling polymer used to fabricate the hydrogel beads is a parameter in determining their physicochemical properties and gastrointestinal fate. Many different gelling biopolymers can be used, including polysaccharides (including but not limited to alginate, carrageenan, agar, pectin, xanthan gum, starch, and chitosan) and proteins (including but not limited to whey protein, caseinate, soy protein, pea protein, legume protein, gelatin, and lactoferrin).

The concentration of biopolymer and cross-linking agent used to fabricate the hydrogel beads is also a parameter determining their physicochemical properties and gastrointestinal fate of the beads. For example, the bead pore size typically decreases and the bead firmness typically increases as the concentration of biopolymer and cross-linking agent increases.

Many different kinds of gelling mechanisms can be used to prepare hydrogel beads depending on the nature of the biopolymers and cross-linking agents used, including physical, chemical, or enzymatic methods, such as heating, cooling, ion addition, electrostatic complexation, aldehyde addition, and enzyme addition.

The diffusion rate of hydrogen ions in and out of the hydrogel beads depends on the dimensions of the hydrogel beads. Typically, the diffusion of small molecules or ions into hydrogel beads can be reduced by increasing the particle diameter, which can be attributed to the longer distance that these substances have to travel to reach the encapsulated functional agent.

The surface properties of the hydrogel beads, such as charge and hydrophobicity, play a role in determining the functional attributes of the beads. Therefore it is often useful to control the surface properties of the hydrogel beads, e.g., by electrostatic deposition of biopolymer coatings onto their surfaces. Another parameter in determining the performance of the hydrogel system is the nature of the encapsulated antacid agents used, which need to be carefully designed for system optimization. Specifically, the internal pH of the hydrogel beads is determined by the nature of the insoluble buffering agents trapped inside them. Either acidic or basic buffering agents can be used depending on the application. For antacid hydrogel beads, the internal pH will be higher if a stronger base is used, e.g., Ca(OH)₂ rather than Mg(OH)₂. Conversely, the internal pH will be lower if a weaker base, e.g., ZnCO₃ rather than Mg(OH)₂. The final microclimate pH in hydrogel beads is also correlated to the amount of encapsulated buffer used.

The invention is further illustrated by the following non-limiting examples.

### EXAMPLES

### Materials and Methods for Examples 1-4

Materials: Fluorescein tetramethylrhodamine dextran (FRD, average Mᵣ 70,000) was purchased from Molecular Probes (Eugene, Oreg.). Alginic acid (sodium salt), calcium chloride, and magnesium hydrate were purchased from the Sigma Chemical Company (St. Louis, Mo.). Sodium phosphate was purchased from Fisher Chemical Company (Pittsburgh, Pa.). All chemicals used were analytical grade. Double distilled water was used to make all solutions.

Hydrogel beads preparation: Aqueous alginate solutions were prepared by dispersing powdered alginic acid (0.8%, w/w) into phosphate buffer solution (5 mM, pH 7.0) and then continuously stirring at 60° C. for an hour. The temperature was then reduced to 35° C. and the samples were continually stirred until the solution became clear indicating that the alginate had fully dissolved. 10 mg/mL FRD in phosphate buffer (5 mM, pH 7.0) was then added to the alginate solution (at a volume ratio of 1:200) with or without 0.3% Mg(OH)₂. After ensuring the sample was homogeneous by stirring, the mixture was injected into a 10% w/w calcium chloride solution using a commercial encapsulation unit (Encapsulator B-390, BUCHI, Switzerland) with a 120 μm vibrating nozzle to prepare the hydrogel beads. The encapsulation device was operated under fixed conditions: frequency=800 Hz; electrode potential=800 V; and operating pressure=500 mbar. The alginate beads formed were kept in the Ca²⁺ solution for 1 h at ambient temperature to promote cross-linking and bead hardening.

Simulated gastric conditions: The impact of exposure of the hydrogel beads to simulated gastric fluid (SGF) on their internal pH was determined using a method described in the art. Hydrogel beads (with or without encapsulated Mg(OH)₂) were added at a ratio of 1:4 (w/w) to SGF that had been preheated to 37° C. and adjusted to pH 2.5. This mixture was then incubated in an incubated shaker for 2 h at 37° C. to mimic stomach conditions.

Confocal Laser Scanning Microscopy: Images were obtained using confocal laser scanning microscopy with a 20× objective lens (Nikon D-Eclipse C1 80i, Nikon, Melville, N.Y., USA). The images of the ratiometric fluorescence dye were obtained using excitation wavelengths of 543 and 488 nm, and detection wavelengths/bandwidths of 650 nm/LP and 590 nm/50 nm, respectively. All samples were imaged using an exposure time of 0.5 s and a 12.5% excitation power level for both channels. The complete images of each sample were typically acquired in less than 2 min with at least eight measurements. The microstructural images for confocal microscopy were analyzed using image analysis software (NIS-Elements, Nikon, Melville, N.Y., USA).

Standard curve preparation: A stock FRD solution (10 mg/mL) was prepared by dissolving the powdered FRD dye in phosphate buffer (5 mM, pH 7) solution. 5 μL/ml FRD stock solution at pH 2.5-7 (adjusted by hydrochloric acid) was imaged using confocal microscopy to determine the linear range of the pH response. Solutions containing different concentrations of FRD (2.5, 5 and 10 μL/ml) were also used to prepare a standard curve using a SpectraMax® spectrophotometer (Molecular Devices, Sunnyvale, Calif.) to assess the impact of dye concentration on the fluorescence intensity ratio used to calculate the local pH.

Image processing for pH measurement: The images were analyzed using Image J Software (1.50I, imagej.nih.gov). The ratio of pixel intensities of two images obtained from two wavelengths (488 and 543 nm) were calculated and correlated with the pH obtained from the standard curve. The average intensity was calculated from at least eight measurements. The pH gradient within the beads was determined by dividing their circular images into a series of same-pixel thickness concentric rings, and the mean pixel intensity was calculated for each ring from the number of pixels and individual pixel intensities in the ring.

### Example 1: Development of pH Standard Curve

In this study, relatively small hydrogel beads (around 200 μm) were fabricated using a specialized extrusion device (Encapsulator) with a vibrating nozzle (FIG. 1). The small dimensions and viscoelastic nature of these beads meant that it was not possible to measure their internal pH using traditional methods, such as pH electrodes. FIG. 2 shows a schematic of the beads. It was therefore necessary to develop an alternative method to measure the pH within the beads so as to confirm the potential of the encapsulated Mg(OH)₂ to control their internal pH profile. A fluorescent probe method was therefore developed to determine the local pH profile inside the beads. A dextran conjugated fluorescent probe, Fluorescein and Tetramethylrhodamine (FRD), was used as the pH indicator. This probe was selected because it has a relatively high molecular weight (around 70 kDa), which ensures that it remains trapped inside the porous biopolymer network that makes up the bead interior. Moreover, this probe is a ratiometric dye that has both pH-dependent (FITC) and pH-independent (TMR) fluorescence groups. For this type of dye, the pH-independent channel can be used as an internal standard for quantitative pH evaluation. This ratiometric method also eliminates problems associated with photo-bleaching, variable bead thickness, and non-uniform dye distribution within the bead.

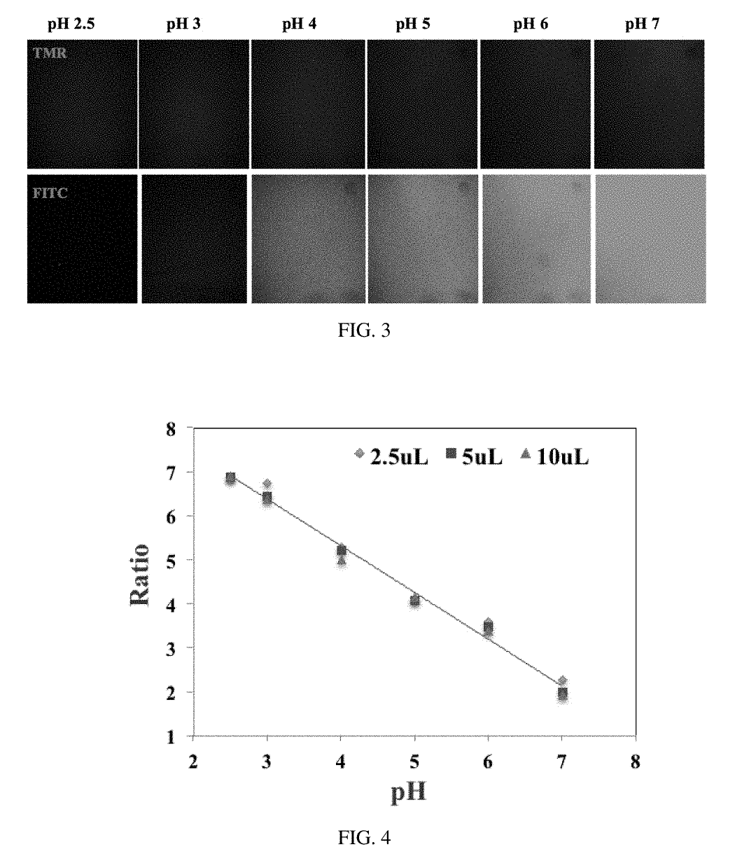

FIG. 3 show fluorescent images of phosphate buffer at pH 2.5, 3, 4, 5, 6, and 7. The images of the first row (TMR channel) and second row (FITC channel) were emitted at 543 and 488, detect at 650/LP and 590/50 respectively.

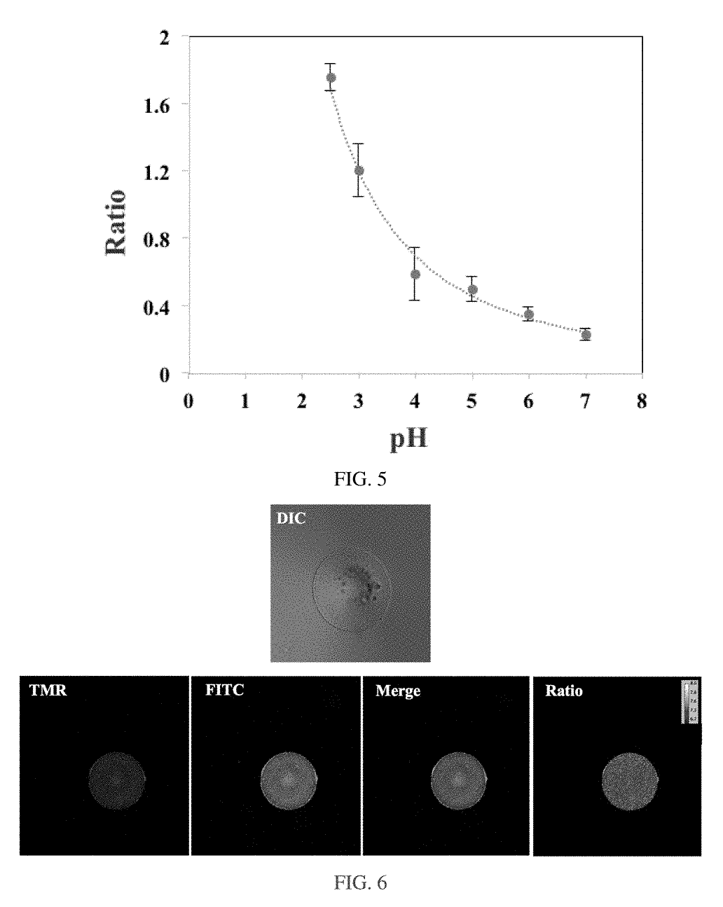

In preliminary confocal microscopy studies, it was observed that the fluorescence emission intensity was pH-independent when an excitation wavelength of 543 nm was used, but pH-dependent when an excitation wavelength of 488 nm was used (data not shown). The pH of a sample could therefore be obtained independent of dye concentration by taking the ratio of the emission intensities at these two excitation wavelengths (543 nm/488 nm). Standard curves of fluorescence intensity ratio versus pH were determined using a series of buffer solutions (pH 2.5-7.0) with dye concentrations of 2.5, 5 and 10 μL/ml of stock solution (10 mg/mL). The fluorescence intensity ratio was exponentially related to pH from pH 2.5 to 7.0 when determined from the confocal microscopy images (FIG. 5), but linearly related when measured using a microplate reader (FIG. 4). Moreover, the fluorescence intensity ratio (TMR/FITC) was largely independent of overall dye concentration across the entire pH range as determined using the microplate reader. These results suggest that the pH inside the beads can be successfully mapped even though there may be some local variations in dye concentration within the beads for example, due to diffusion of dye outwards. The major advantage of the confocal microscopy approach is that the local pH can be obtained from the images, whereas only the overall pH of a sample can be obtained using the microplate reader.

### Example 2: Encapsulation of (Mg(OH)₂) within Hydrogel Beads

The highly acidic pH of human gastric juices causes an unwanted reduction in the activity of many orally delivered bioactive agents, such as many enzymes, probiotics, and nutraceuticals. Consequently, an antacid buffering agent (Mg(OH)₂) was encapsulated inside the hydrogel beads to create an internal pH that was considerably higher than the external pH, thereby protecting any co-encapsulated acid-sensitive bioactive agents. Under neutral pH conditions, Mg(OH)₂ forms insoluble solid particles that are retained within the hydrogel beads. However, when the beads are exposed to acid conditions some of the Mg(OH)₂ particles will dissolve, thereby releasing OH⁻ ions that will neutralize any H⁺ ions that diffuse into the hydrogel interior, thereby helping to maintain a high internal pH.

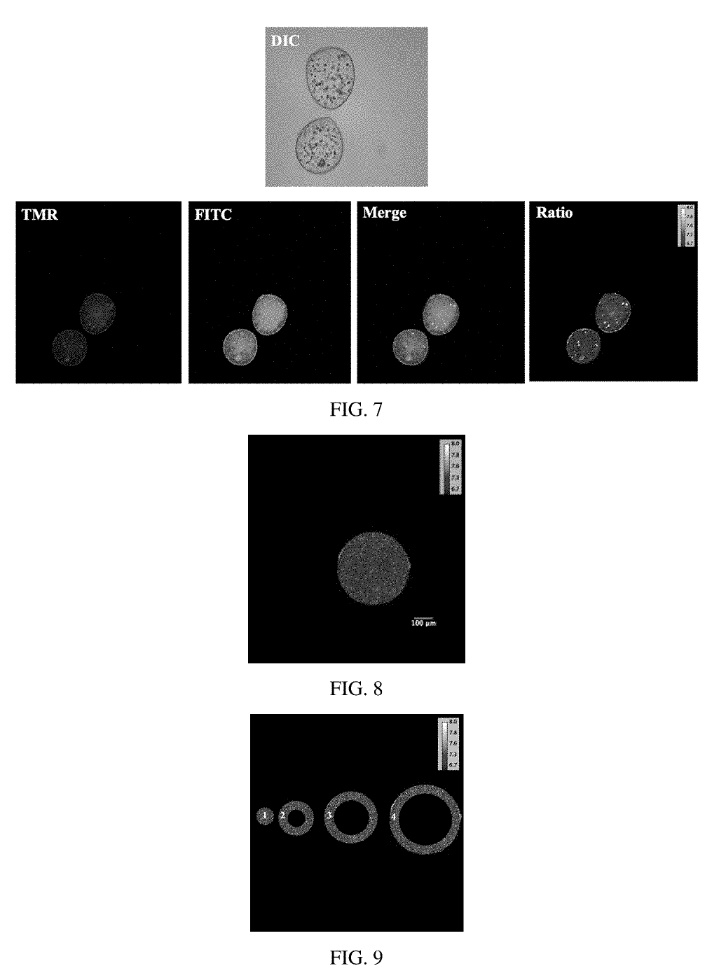

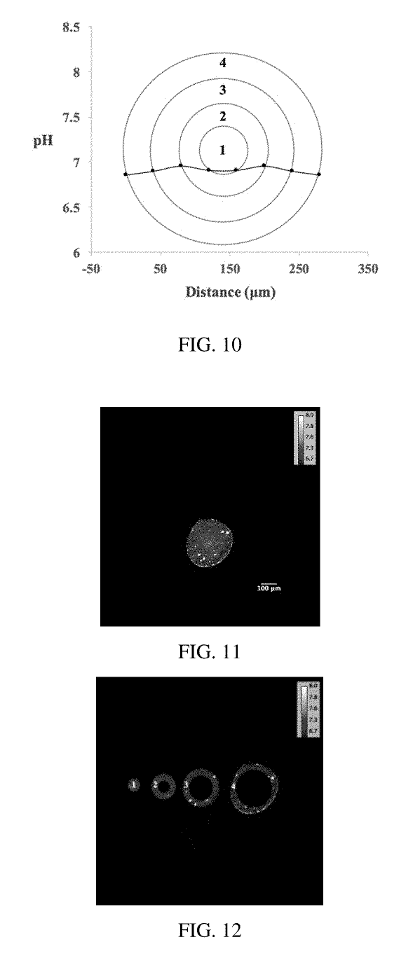

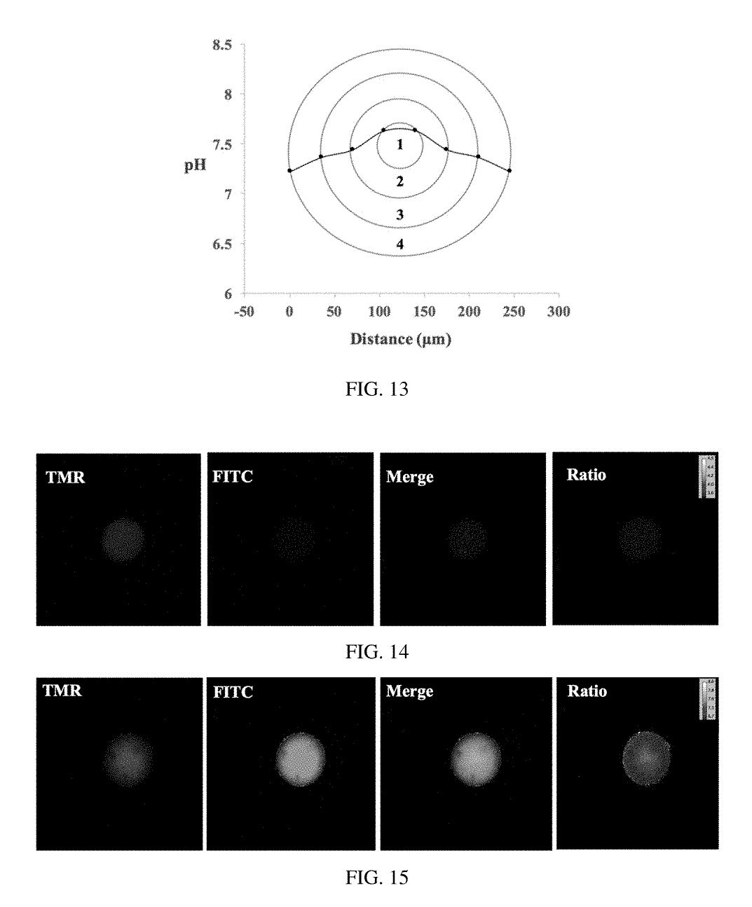

Hydrogel beads were formed by injecting a solution containing gelling agent (alginate) and fluorescence dye (FRD) in the absence or presence of insoluble buffer particles (magnesium oxide) into another solution containing a cross-linking agent (calcium chloride). The initial beads formed by this method had mean diameters of around 200 μm as determined by confocal fluorescence and optical microscopy (FIG. 6-13). Initially, the average internal pH values of the hydrogel beads determined from the CLSM images were around pH 6.5 for buffer-free beads and around pH 7.5 for buffer-loaded beads. The presence of Mg(OH)₂ slightly increased the internal pH of the buffer-loaded hydrogel beads, which can be attributed to the fact that it is a weak base and some dissolution may have occurred. The confocal images also indicated that the pH values inside the beads were not uniformly distributed after Mg(OH)₂ encapsulation (FIG. 7). There appeared to be microenvironments around the insoluble Mg(OH)₂ particles that had a higher pH that the surrounding solution. The pH gradient distribution analysis indicated that the pH was relatively uniform throughout the buffer-free hydrogel beads, being in the range from pH 6.8-7 (FIG. 10). Conversely, the pH was slightly lower at the edge of the buffer-loaded hydrogel beads than in their interiors, with the local pH values increasing from around pH 7.2 at the edge to pH 7.6 at the center (FIG. 13). Presumably, most of the insoluble buffer particles were located in the center rather than at the edge of the hydrogel beads.

### Example 3: Monitoring pH Changes after Exposure to Gastric Conditions

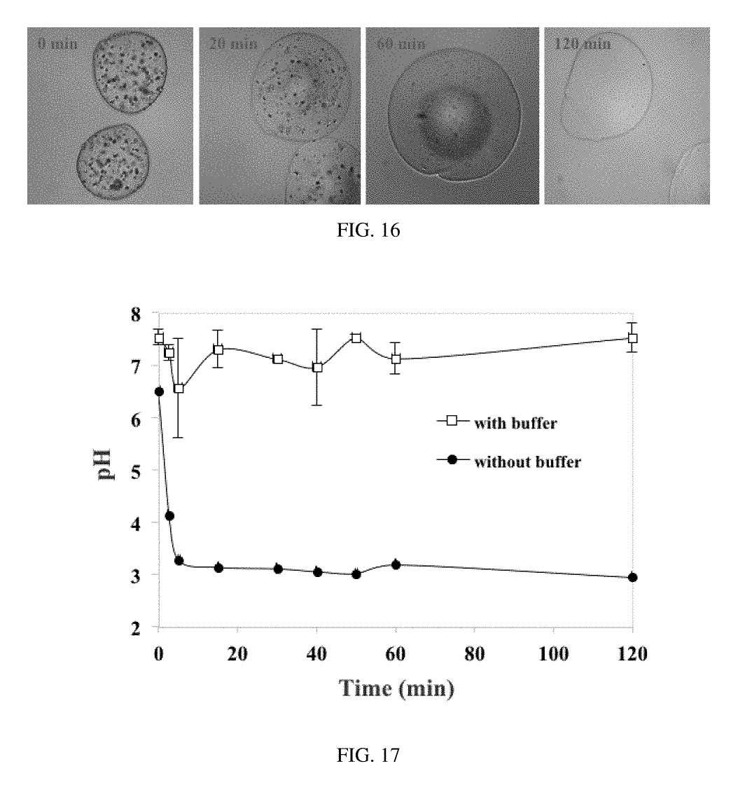

Many types of bioactive agents are degraded when exposed to the highly acidic conditions found in the human stomach, and therefore it would be valuable to have delivery systems that protected them within the gastric fluids. In this section, we therefore applied the confocal fluorescence pH mapping method to monitor changes in the internal pH of the hydrogel beads in the absence and presence of encapsulated magnesium hydroxide. After incubation in simulated gastric fluids (SGF), the fluorescence intensity inside the buffer-free hydrogel beads rapidly decreased to around pH 3 (FIG. 14). Presumably, the small H⁺ ions rapidly diffused through the porous alginate network within the hydrogel beads driven by the concentration gradient between the inside and outside of the beads. Consequently, the internal pH of the buffer-free beads was fairly similar to that of the external pH of the SGF after 2 h incubation in the gastric phase. Conversely, the pH value within the Mg(OH)₂-loaded hydrogel beads remained fairly similar to that of the original value (approximately pH 7.5) after 2 hours of incubation in the acidic SGF (FIG. 15). This result suggests that these antacid hydrogel beads could be used to protect acid-sensitive bioactive agents during passage though the stomach. The confocal fluorescence images suggest that the pH was higher in the center of the beads than at their edges (FIG. 15). This effect is probably because the insoluble Mg(OH)₂ particles in the exterior regions of the beads dissolve first as hydrogen ions from the SGF diffuse into the beads. To verify this dissolution process, DIC optical images of the Mg(OH)₂-loaded hydrogel beads were taken throughout the simulated stomach phase (FIG. 16). These images clearly indicated that Mg(OH)₂ particles slowly dissolved during the stomach phase incubation, which can be attributed to their neutralization reaction with hydrogen ions arising from the gastric fluids.

### Example 4: Dynamic pH Change During Incubation

The acid degradation of many bioactive agents is a rapid process, and so it is important to determine how long the hydrogel beads can maintain their high internal pH values after exposure to gastric fluids. Therefore, the internal pH of buffer-free and buffer-loaded hydrogel beads was periodically determined throughout the stomach phase (FIG. 17). In the presence of buffer, the average pH inside the beads remained fairly constant (pH 6.6 to 7.5) throughout the entire 2 hour incubation period within the SGF (FIG. 17). This result suggested that the amount of Mg(OH)₂ inside the beads was sufficient to act as a local antacid throughout the duration of the gastric phase. On the contrary, the pH value inside the buffer-free hydrogel beads fell sharply from pH 6.5 to 3.3 within the first 5 minutes, followed by a more gradual decrease at longer incubation times. As mentioned earlier, small H⁺ ions can rapidly diffuse into highly porous hydrogel beads, thereby causing the pH inside the beads to be fairly similar to that outside.

The amount of time required for hydrogen ions to diffuse into the hydrogel beads can be estimated from the following expression:

$$Φ = \frac{M ( t )}{M ( ∞ )} = 1 - \frac{6}{{π}^{2}} {\sum }_{n = 1}^{∞} \frac{1}{{n}^{2}} exp ( - \frac{{D}_{gel} {n}^{2} {π}^{2} t}{{a}^{2}} ) \qquad ( 1 )$$

Here, Φ is the fraction of hydrogen ions that have diffused into the hydrogel beads at time t, M(t) and M(∞) are the hydrogen ion concentrations within the hydrogel beads at time t and at equilibrium, n is an integer, a is the radius of the hydrogel beads, and D_{gel} is the diffusion coefficient of the hydrogen ions through the biopolymer network inside the beads, which can be estimated using the following expression (Chan & Neufeld, 2009):

$${D}_{gel} = {D}_{w} exp ( - π ( \frac{{r}_{H} - {r}_{f}}{ξ + 2 {r}_{f}} ) ) \qquad ( 2 )$$

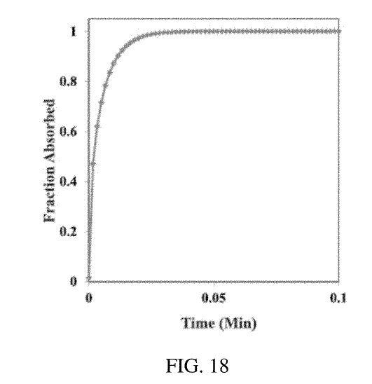

Here, D_{w} is the diffusion coefficient of the hydrogen ions through pure water, r_{H} is the hydrodynamic radius of the hydrogen ions, r_{f} is the cross-sectional radius of the biopolymer chains (alginate) in the hydrogel network, and ζ is the mesh pore diameter of the hydrogel network. The following structural parameters have been reported for alginate hydrogels: pore diameter (ζ)=4 to 400 nm; alginate chain radius (r_{f})=0.83 nm. The radius of hydration (r_{H}) of the hydrogen ion (actually H₃O⁺) has been estimated to be 0.1 nm. The diffusion coefficient of hydrogen ions in water can be estimated from the expression: D_{w}=k_{B}T/6πηr_{H}, where k_{B} is Boltzmann's constant, T is absolute temperature, and η is the viscosity of the solvent. The above equations were used to estimate the time-dependence of the fraction of hydrogen ions diffusing into hydrogel beads with different pore sizes after submerging in a more acidic environment (FIG. 18). For these calculations, it was assumed that the pore diameter (ζ) of the alginate beads was 10 nm. Even assuming this relatively small pore size, the theoretical predictions suggest that the hydrogen ions would rapidly diffuse into the hydrogel beads in a few seconds, which supports our experimental results. These predictions suggest that any acid-sensitive bioactives encapsulated within the hydrogels beads would be rapidly exposed to highly acid conditions in the stomach in the absence of a co-encapsulated insoluble buffering agent.

### Conclusions: Examples 1-4

Certain types of bioactive agents are degraded when exposed to highly acidic conditions, such as probiotics, enzymes, and some nutraceuticals. The development of microparticle-based delivery systems that are able to control the pH in the immediate vicinity of these bioactive agents may therefore be useful for improving their stability. In this study, edible hydrogel beads were fabricated using a simple injection method that were able to maintain an internal pH that was appreciably higher than the pH of the surrounding solution. A fluorescence method to map the pH profile inside the hydrogel beads was also developed based on ratiometric imaging with a confocal laser scanning microscopy. This method showed that the average internal pH of the buffer-free hydrogel beads was around 6.5, while that of the buffer-loaded beads was around 7.5. In addition, the method showed that there were some local variations in pH inside the hydrogel beads. After exposure to simulated gastric fluids, the average pH inside the buffer-loaded beads remained fairly constant (around pH 6.6 to 7.5), whereas that in the buffer-free beads fell sharply within the first few minutes (from pH 6.5 to 3.3). The confocal microscopy method also indicated that the pH was higher in the center of the buffer-loaded beads than at their edges. These results show that the encapsulated Mg(OH)₂ particles acted as effective buffering agents that inhibited excessive acidification within the beads during the gastric phase. Overall, the novel hydrogel beads developed in this study may be suitable for encapsulation and delivery of acid-sensitive bioactive agents, which may be advantageous for certain food, supplement, and pharmaceutical applications.

### Materials and Methods for Example 5

Materials: Fluorescein tetramethylrhodamine dextran (FRD, average Mᵣ 70,000) was obtained from Molecular Probes (Eugene, Oreg.). Alginic acid (sodium salt), calcium chloride, and magnesium hydroxide were purchased from Sigma Chemical Company (St. Louis, Mo.). Lipase was purchased from Sigma-Aldrich (Sigma Chemical Co., St. Louis, Mo.) and as reported by the manufacturer the activity was 100-400 units/mg. Mucin from porcine stomach, porcine bile extract, sodium chloride, monobasic phosphate and dibasic phosphate, Nile red and fluorescein thiocyanate isomer I (FITC) were obtained from either Sigma-Aldrich (Sigma Chemical Co., St. Louis, Mo.) or Fisher Scientific (Pittsburgh, Pa.). All chemicals used were analytical grade. Double distilled water was used to make all solutions.

Hydrogel beads preparation: An aqueous alginate solution was prepared by dissolving powdered alginic acid (1.6%, w/w) in phosphate buffer and continuously stirring at 60° C. for an hour, then reducing the temperature to 35° C. with continuous stirring until fully dissolved. The alginate solution was then mixed with lipase solution to obtain a concentration of 0.8% alginate and 2.7% lipase mixture with or without 0.15% Mg(OH)₂. For the pH mapping, 10 mg/mL FRD in phosphate buffer was added to 0.8% alginate solutions (at a volume ratio of 1:200) with or without 0.15% Mg(OH)₂. After continuously stirring, the mixtures were injected into 10% calcium chloride solution using a commercial encapsulation unit (Encapsulator B-390, BUCHI, Switzerland) with a 120 mm vibrating nozzle to prepare the hydrogel beads. The encapsulation device was operated under fixed conditions: frequency 800 Hz; electrode 800 V; and pressure 500 mbar. The formed beads were held in the Ca²⁺ solution for 30 min at ambient temperature to promote alginate cross-linking and bead hardening.

In vitro digestion model: The lipase-loaded hydrogel beads with or without co-encapsulated buffer (0.15% Mg(OH)₂) were passed through a simulated gastrointestinal tract (GIT) that included mouth, stomach and small intestine phases, which was slightly modified from one previously used. A control sample containing non-encapsulated lipase was also studied under similar simulated GIT conditions.

Initial System:

7.5 g of the initial systems were placed into a glass beaker in an incubator shaker at a rotation speed of 100 rpm for 15 min at 37° C.

Mouth Phase:

7.5 g of simulated saliva fluid (SSF) containing 0.03 g/mL mucin was preheated to 37° C. and then adjusted to pH 6.8. After being mixed with the initial samples, the mixture was incubated in an incubator shaker for 2 min at 37° C. to mimic agitation in the mouth.

Stomach Phase:

15 g of simulated gastric fluid was preheated to 37° C., and then the pH was adjusted to 2.1. After being mixed with 15 g of the sample resulting from the mouth phase, the initial pH of the mixture was about 2.5 and incubated in the incubator shaker for 2 h at 37° C. to mimic stomach conditions.

Small Intestine Phase:

30 g of sample resulting from the stomach phase was placed into a 100 mL glass beaker that was placed into a water bath at 37° C. and then adjusted to pH 7.00. 1.5 g of simulated intestinal fluid was added to the reaction vessel, followed by 3.5 g of bile salt solution with constant stirring. The pH of the reaction system was adjusted back to 7.00. Then, 2.5 g of 6% (w/w) corn oil-loaded emulsions were added to the sample and an automatic titration unit (Metrohm, USA Inc.) was used to monitor the pH and maintain it at pH 7.0 by titrating 0.1 N NaOH solution into the reaction vessel for 2 h at 37° C. The amount of free fatty acids released was calculated from the titration curves using the following formula:

$$\% FFA = 100 \times ( \frac{{V}_{NaOH} \times {m}_{NaOH} \times {M}_{Lipid}}{{W}_{Lipid} \times 2} ) \qquad ( 3 )$$

Here V_{NaoH} is the volume of sodium hydroxide solution required to neutralize the FFAs produced (mL), m_{NaoH} is the molarity of the sodium hydroxide solution (0.1 N), W_{Lipid} is the total weight of lipid initially present in the reaction vessel (0.15 g), and M_{Lipid} is the molecular weight of the corn oil (800 g/mol).

Confocal Laser Scanning Microscopy: Images were obtained using a confocal scanning laser microscope with a 20× objective lens (Nikon D-Eclipse C1 80i, Nikon, Melville, N.Y., USA). The confocal images of the ratiometric dyes used were obtained using 543 and 488 nm excitation wavelengths, and 650 nm/LP and 590 nm/50 emission wavelengths, respectively. All samples were imaged with an exposure time of 0.5 s and a 12.5% excitation power level for both channels. The images for each sample were typically acquired in less than 2 min with at least eight measurements per sample. After incubation under small intestine conditions, the oil phase of the samples was dyed with Nile red solution (1 mg/mL ethanol). In addition, the lipase was dyed using FITC solution (1 mg/mL dimethyl sulfoxide) prior to measurements by incubating the lipase-loaded beads in 0.1 mL of FITC dye solution. The excitation and emission spectrum for Nile red were 543 nm and 605 nm, respectively and for FITC were 488 nm and 515 nm, respectively. The microstructure images acquired by confocal microscopy were stored and analyzed using image analysis software (NIS-Elements, Nikon, Melville, N.Y., USA).

Standard curve preparation: A stock FRD solution (10 mg/mL) was prepared by dissolving powdered FRD dye in phosphate buffer (5 mM, pH 7) solution. 5 μL/ml FRD stock solution at pH 4-7 (adjusted by hydrochloric acid) was imaged using confocal microscopy to determine the linear range of pH response.

Image processing for pH measurement: The confocal microscopy images were analyzed using Image J software (1.50I, imagej.nih.gov). The ratio of pixel intensities of two images obtained from two wavelengths (488 nm, 543 nm) were calculated and correlated with pH from the obtained standard curve. The images were processed by repeated scans with frame averaging from at least eight measurements.

Determination of droplet size: The particle size distribution of the hydrogel beads with and without co-encapsulated Mg(OH)₂ were measured using laser diffraction (Mastersizer 2000, Malvern Instruments Ltd., Malvern, Worcestershire, UK), which is based on analysis of the angular scattering pattern of particulate suspensions. Samples were diluted in aqueous solutions to avoid multiple scattering effects, and then stirred (1200 rpm) to ensure homogeneity. Phosphate buffer (5 mM, pH 7.0) was used to dilute the initial samples, while pH 2.5 adjusted distilled water was used to dilute gastric samples. The average particle sizes are reported as the volume-weighted mean diameter (d₄₃).

Statistical analysis: All experiments were performed on at least three freshly prepared samples. The results are reported as means and standard deviations. Statistical analyses were carried out using Excel (Microsoft, Redmond, Va., USA) and statistical differences (p<0.05) were established using a statistical software package (SPSS).

### Example 5: Encapsulation of Lipase in the Hydrogel Beads with Self-Regulating Microclimate pH Properties for the Enzyme Activity Reservation During Digestion

Exocrine pancreatic insufficiency (EPI) is a serious condition that accompanies several diseases, including chronic pancreatitis and pancreatic cancer. EPI occurs when the pancreas does not generate and/or secrete sufficient amounts of digestive enzymes such as lipases, proteases, and amylases) to appropriately hydrolyze ingested lipids, proteins and carbohydrates, which results in nutritional deficiencies. Lipase is one of the most important digestive enzymes derived from the pancreas and it plays a leading role in the digestion of lipids, which typically provide the majority of calories to the human body. In addition, lipid digestion and mixed micelle formation in the small intestine is usually an important precursor to the absorption of lipophilic vitamins and nutraceuticals. Enzyme supplementation is an important therapy used to treat individuals suffering from pancreatic insufficiency. However, the supplementation of lipase through the oral route is often ineffective because this enzyme is highly susceptible to degradation during passage through the highly acidic environment of the stomach. In healthy individuals, it has been reported that only 1% of lipase activity is retained after passage through the stomach. For this reason, there is considerable interest in the development of effective delivery systems that can encapsulate lipase, protect it in the stomach, and then rapidly release it in the small intestine where it can digest any lipids. These delivery systems could then be ingested at the same time as a fat-containing meal to increase calorie intake and bioactive absorption.

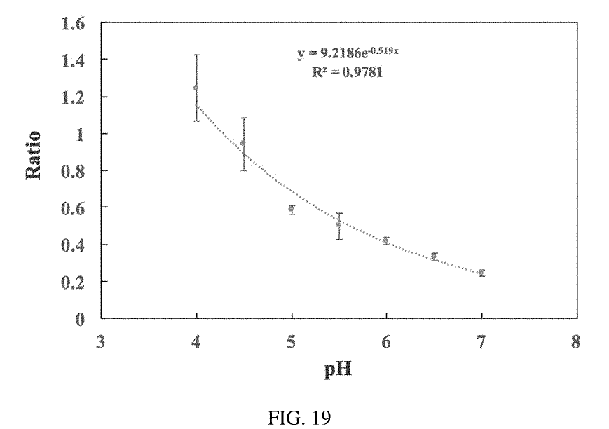

In this study, a standard curve of fluorescence intensity ratio versus pH was established using buffer solutions covering the range pH 4 to 7 (FIG. 19). The standard curve clearly shows that the intensity ratio decreases with decreasing pH, and can therefore be used to measure pH in this range.

An insoluble antacid buffer agent (Mg(OH)₂) was encapsulated within the hydrogel beads so as to control the internal pH inside the beads throughout the simulated GIT process. Mg(OH)₂ was used because it will be retained as solid particles inside the hydrogel beads under neutral conditions, but will partly dissolve and release hydroxide ions (OH⁻) when exposed to acid conditions. Hydrogel beads fabricated using similar preparation conditions, but in the absence of Mg(OH)₂, were studied as control samples. The pH values inside hydrogel beads with or without Mg(OH)₂ were then measured after incubation under different simulated GIT conditions (initial, mouth, stomach, and small intestine) using the standard curve described in the previous section (FIG. 19).

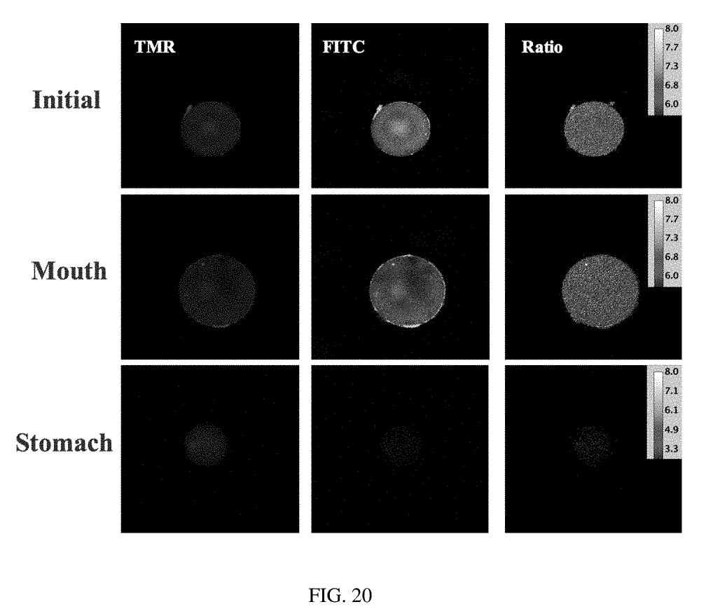

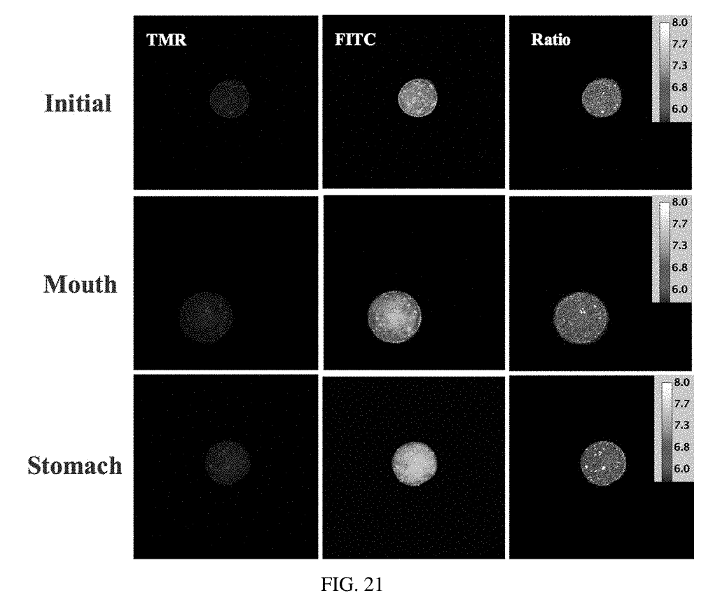

Confocal microscopy images of the hydrogel beads using the FITC and TMR channels highlighted changes in their internal pH in different regions of the GIT (FIGS. 20 and 21). The pH value inside the hydrogel beads remained fairly constant for the initial samples and the samples after incubation in the mouth phase, irrespective of Mg(OH)₂ encapsulation, as shown by the fact that the TMR channel shows that the bead interior had a relatively strong fluorescence intensity. This result can be attributed to the fact that the pH in the initial phase and the mouth phase were fairly similar (pH 7 and 6.8, respectively). After exposure to the stomach phase, the fluorescence intensities of the hydrogel beads containing Mg(OH)₂ measured using both the FITC and TMR channels remained relatively strong, which is indicative of a high internal pH. Conversely, the fluorescence intensities from both the FITC and TMR channels were very weak for the hydrogel beads without buffering agent, which suggested that the pH was very low inside the beads.

The average internal pH values of the hydrogel beads calculated from the calibration curve are shown in Table 2. Initially, the internal pH of the hydrogel beads was higher for the beads containing buffer (pH 7.25) than for those without buffer (pH 6.74), which would be expected due to the fact that a small amount of the Mg(OH)₂ may have dissolved and released some hydroxyl ions. After exposure to simulated mouth conditions, the internal pH of the beads remained fairly close to neutral, i.e., pH 6.29 for buffer-free beads and pH 7.33 for buffer-loaded beads. The microscopy ratio images indicated that the fluorescence intensity was uniformly distributed within the buffer-free beads, which indicated that the pH was also relatively uniform throughout the beads matrix (FIG. 20). Conversely, for the buffer-loaded beads, the fluorescence intensity was more unevenly distributed, which can be attributed to a higher local pH around the insoluble Mg(OH)₂ particles (FIG. 21). After incubation in simulated gastric fluids, the fluorescence intensity sharply decreased for the buffer-free hydrogel beads, which suggested that the pH was below the limit of detection (pH<4) based on the calibration curve (FIG. 19). This result can be attributed to the fact that small hydrogen ions can rapidly diffuse through the pores of the hydrogel beads leading to a low internal pH value. On the contrary, the pH inside the buffer-loaded beads remained relatively high and close to neutral (pH 7.39) after exposure to the simulated gastric fluids. These results suggest that some of the Mg(OH)₂ particles slowly dissolved and released hydroxyl ions that were able to neutralize the hydrogen ions arising from the gastric fluids, thereby maintaining the neutral pH conditions inside the beads. The buffer-loaded beads contain about 1.5 mg/mL of Mg(OH)₂ (i.e., 0.15 w/w %), which corresponds to about 51 mM of OH⁻ inside the beads. The confocal microscopy images were taken after 2 hours submersion of the hydrogel beads in simulated gastric fluids, which suggests that there was sufficient buffer present within the beads to maintain the internal pH over the time foods, supplements, or drugs normally spend in the stomach.

TABLE 2
The measured pH value inside beads with/without
buffer encapsulation during digestion process

| | Initial | Mouth | Stomach |
|---|---|---|---|
| Without buffer (pH) | 6.74 ± 0.08 | 6.29 ± 0.104 | Acid pH |
| With buffer (pH) | 7.25 ± 0.106 | 7.33 ± 0.182 | 7.39 ± 0.15 |

The influence of encapsulation on the ability of lipase to carry out lipid digestion was studied using an automatic titration (“pH-stat”) method. Lipase-loaded beads, with or without co-encapsulated Mg(OH)₂, were passed through the mouth and stomach phases, and then they were incubated with emulsified lipids (corn oil-in-water emulsion) under simulated small intestine conditions. The amount of free fatty acids released over time was then calculated from the volume of NaOH added to the samples so as to maintain a constant pH value (7.0). A control experiment was carried out by exposing free lipase (no hydrogel beads) to the same simulated GIT conditions.

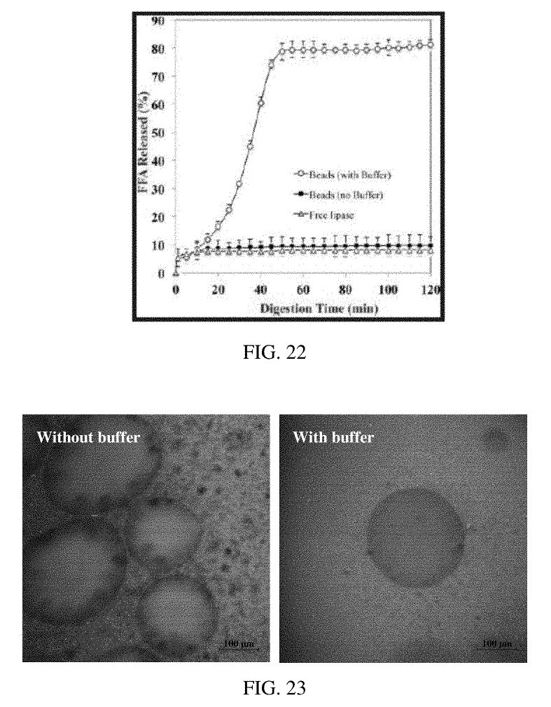

For the free lipase and the lipase-loaded buffer-free hydrogel beads, there was almost no free fatty acids released throughout the entire small intestine phase, which suggested that the lipase had been deactivated in the stomach digestion process (FIG. 22). The loss of enzymatic activity of lipases upon exposure to low pH conditions has been attributed to the titration of the active site histidine or to the weakening of the coordination of stabilizing calcium ions. These results suggest that the encapsulation of lipase in alginate beads on its own was insufficient to protect the lipase from acid conditions. As mentioned earlier, this effect can be attributed to the fact that small hydroxyl ions (H⁺) can easily diffuse into beads incubated in simulated gastric fluids, thereby lowering the internal pH and inactivating the lipase, as suggested by the confocal microscopy measurements (FIG. 20, Table 2). The lipid digestion profile of the lipase-loaded beads containing co-encapsulated Mg(OH)₂ was appreciably different from the buffer-free beads (FIG. 22). During the first 10 minutes, there was a delay in the generation of free fatty acids, which can be attributed to the time required for lipase molecules to diffuse through the beads and into the surrounding medium. After this time, there was a rapid increase in free fatty acids release up to about 45 min, after which there was a more gradual increase until a relatively constant final value was attained. These results indicate that the activity of lipase was preserved when it was co-encapsulated with Mg(OH)₂ in the hydrogel beads.

Further evidence of the ability of the buffer-loaded beads to retain and stabilize lipase was obtained using confocal microscopy (FIG. 23). Lipase was dyed green using FITC, whereas the oil phase was dyed red using Nile red. The confocal microscopy images indicate that most of the lipase was trapped inside the hydrogel beads, i.e., the green (lipase) fluorescence dye was mostly located in the bead interior (FIG. 23). This result suggests that the majority of lipase was successfully delivered to the small intestine phase using the hydrogel beads. Some green fluorescence intensity could also be detected outside the beads in the simulated intestinal fluids, which suggests that some of the lipase was released from the beads. Presumably, the released lipase was able to adsorb to the surfaces of the emulsified lipids and promote their hydrolysis into free fatty acids. Alginate beads have previously been reported to swell when placed in neutral pH solutions because anionic groups on the alginate molecules (carboxylate groups) become highly charged and repel each other causing the hydrogel network to expand. The relatively large pore size of swollen beads may have promoted the release of lipase. There were also pronounced differences in the nature of the lipid phase after exposure to small intestine conditions depending on whether the beads contained buffer or not (FIG. 23). For the buffer-free hydrogel beads, the emulsion remained as small individual lipid droplets that surrounded the beads and were difficult to observe in the confocal images because their dimensions were close to the limit of resolution of the microscope. Conversely, for the buffer-loaded hydrogel beads, the initial emulsion was converted into large irregular shaped lipid-rich particles, which is characteristic of the mixed micelles formed by lipid digestion. This result also suggests that encapsulation of lipase in the buffer-loaded beads maintained its activity throughout the GIT.

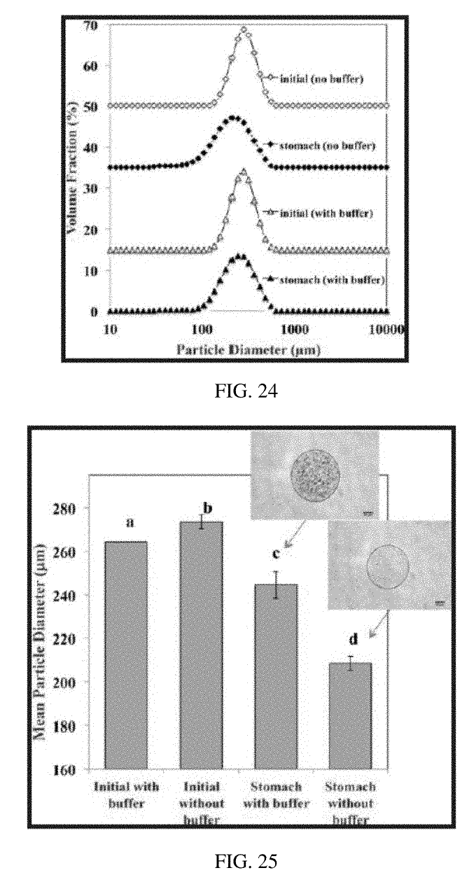

As mentioned earlier, alginate beads may swell or shrink depending on their pH relative to the pKₐ value of their charged groups. Calcium alginate tends to shrink under highly acidic conditions because of the reduction in the electrostatic repulsion between the polymer chains when the carboxyl groups become protonated (—COOH, pKₐ=3.5). Conversely, they tend to swell at higher pH values because of the electrostatic repulsion between the negatively charged polymer chains. It is therefore interesting to study the impact of external pH on the dimensions of the buffer-free and buffer-loaded hydrogel beads within the simulated GIT. The particle size distribution and mean particle diameter of the hydrogel beads were measured using static light scattering (FIGS. 24 and 25). For the initial samples, the mean diameter (d₄₃) of the buffer-loaded beads (264 μm) was slightly lower than that of the buffer-free beads (274 μm), which suggested that the presence of the magnesium hydroxide may have caused some shrinkage of the alginate network. Partial dissolution of the buffer may have released some Mg²⁺ ions that increased the cross-linking of the alginate molecules. Exposure to stomach conditions caused both types of beads to shrink, but the effect was appreciably larger for the buffer-free beads (208 μm) than for the buffer-loaded beads (245 μm). This difference can be attributed to the fact that the internal pH of the buffer-loaded beads is higher than that of the buffer-free beads, and therefore one would expect the alginate molecules would have a stronger negative charge and greater electrostatic repulsion.

### Conclusions from Example 5

Individuals who suffer from exocrine pancreatic insufficiency benefit from the availability of effective oral delivery systems that encapsulate lipase, protect it from acid degradation in the stomach, and then release it in an active form in the small intestine. In this study, lipase was encapsulated within calcium alginate hydrogel beads fabricated using an injection-gelation method. Beads were fabricated in the absence or presence of a buffering agent (Mg(OH)₂) that is insoluble under neutral conditions, but soluble under acidic conditions. A fluorescence method based on laser scanning confocal microscope was used to measure changes in the internal pH of the beads when they were exposed to simulated gastrointestinal conditions. For buffer-loaded beads, the internal pH remained fairly constant (around neutral) upon exposure to mouth or stomach conditions. Conversely, for buffer-free beads, the internal pH decreased appreciably from mouth (around neutral) to stomach (pH<4). As a result, lipase encapsulated in buffer-loaded beads maintained its enzymatic activity in the small intestine, whereas lipase encapsulated in buffer-free beads lost all of its activity. The internal pH of the hydrogel beads also impacted their swelling/shrinking behavior in simulated GIT fluids. The higher pH in the interior of the buffer-loaded beads meant that they swelled less than the buffer-free beads under simulated gastric conditions. The Mg(OH)₂-loaded beads developed in this study may be useful for protecting lipase and other acid-labile substances.

### Materials and Methods for Example 6

Materials: Fluorescein tetramethylrhodamine dextran (FRD) with an average molecular weight of about 70 kDa was purchased from Molecular Probes (Eugene, Oreg.). Lactase (β-galactosidase) with a specific activity of around 2600 U/g was obtained from Sigma Chemical Co (St. Louis, Mo., USA). Carrageenan was kindly donated by FMC Biopolymer (Viscarin SD 389, Philadelphia, Pa.). The reagents o-nitrophenol (o-NP) and o-nitrophenyl-b-d-galactosidase (o-NPG) were obtained from the Sigma Chemical Co. (St. Louis, Mo., USA). All chemicals used were analytical grade. Double distilled water was used to prepare all solutions.

Hydrogel beads preparation: An aqueous κ-carrageenan (2% w/v) solution was prepared by dissolving the powdered ingredient in distilled water by stirring at 50° C. for an hour, and then reducing the temperature to room temperature with continuous stirring until fully dissolved. The κ-carrageenan solution was then mixed with β-galactosidase (β-gal) solution to obtain a concentration of 1% κ-carrageenan and 130 U β-gal mixture with or without different amount of Mg(OH)₂ co-encapsulation. After continuously stirring, the mixtures were injected into 10% potassium chloride solution using a syringe or a commercial encapsulation unit (Encapsulator B-390, BUCHI, Switzerland) with a 150 μm vibrating nozzle to prepare the hydrogel beads. The encapsulation device was operated under the following conditions: frequency 800 Hz; electrode 750 V; and pressure 450 mbar. The formed beads were held in the K⁺ solution for 30 min at ambient temperature to promote bead hardening.

Simulated gastric conditions: A simulated stomach model was used to investigate the influence of exposure to acidic gastric fluids on the internal pH and lactase activity of the hydrogel beads. Hydrogel beads (with or without Mg(OH)₂) were added to preheated simulated gastric fluids (pH 2.5, 37° C.) at a ratio of 1:4 (w/w). This mixture was then incubated in an incubated shaker for 2 h at 37° C. to mimic stomach conditions.

Local pH Determination by Confocal Laser Scanning Microscopy: The fluorescence images were obtained using confocal laser scanning microscopy with a 20× objective lens (Nikon D-Eclipse C1 80i, Nikon, Melville, N.Y., USA). The images of the fluorescence probe (FRD) were obtained using emission wavelengths of 543 and 488 nm, and detection wavelengths/bandwidths of 650 nm/LP and 590 nm/50 nm, respectively. All samples were imaged using an exposure time of 0.5 s and a 12.5% excitation power level. The complete images of each sample were typically acquired in less than 2 min with at least eight measurements. The microstructural images for confocal microscopy were acquired using image specialized software (NIS-Elements, Nikon, Melville, N.Y., USA).

A stock FRD solution (10 mg/mL) was prepared by dissolving powdered FRD dye in phosphate buffer (5 mM, pH 7) solution. 5 μL/ml of FRD stock solution was dissolved in a series of phosphate buffer solutions with different pH values and imaged using confocal microscopy to determine the linear range of pH response.

The confocal microscopy images were analyzed using Image J software (1.50I, imagej.nih.gov). The ratio of pixel intensities of images taken using two wavelength channels (488 nm, 543 nm) was calculated and correlated to the pH using the standard curve. Each pH value was estimated from at least eight individual measurements.

Measurement of lactase activity: The relative activity of β-gal encapsulated in hydrogel beads that was retained after different incubation times in simulated gastric fluids was assayed using a colorimetric test with o-NPG as a substrate. Filtered beads were added to 2 mL of o-NPG in phosphate buffer (0.3 mg/ml) to reach a final alginate to o-NPG ratio of 1:40 (v/v). The formation rate of free o-NP was recorded by measuring the absorbance (λ=420 nm) of solutions contained in a 1-cm path length cuvette using a UV-visible spectrophotometer. The relative activity of encapsulated lactase was calculated based on the conversion rate of the substrate (o-NPG) into the reaction products (o-NP) initially and after stomach phase incubation. At least three freshly prepared samples were used for the activity measurements.

Measurement of bead dimensions: The particle size distribution of the small beads was measured using laser diffraction (Mastersizer 2000, Malvern Instruments Ltd., Malvern, Worcestershire, UK), which is based on analysis of the angular scattering pattern of particulate suspensions. Samples were diluted in aqueous buffer solutions to avoid multiple scattering effects, and then stirred (1200 rpm) to ensure homogeneity. The diameter of the large beads was measured using a digital micrometer (0-300 mm, EC10, High Precision Digital Caliper, Tresna Instruments, Guilin, China). The bead diameter of at least 5 individual beads was measured, and the mean and standard deviation were calculated from at least three measurements.

Statistical analysis: All experiments were performed on at least three freshly prepared samples. The results are reported as means and standard deviations calculated using Excel (Microsoft, Redmond, Va., USA). ANOVA analysis was carried out using a statistical software package (SPSS).

### Example 6: Influence of Encapsulation on Lactase Enzyme Activity

Lactose intolerance is a common disease in humans that is associated with the deficiency of a specific enzyme (β-galactosidase) produced within the brush border of the small intestine. Individuals with lactose intolerance have a limited ability to digest and absorb lactose in the small intestine. As a result, this disaccharide enters the colon in an undigested form where it can promote health problems, such as gut distension, stomach pain, flatulence, diarrhea, and nausea. Individuals who suffer from lactose intolerance often avoid consuming milk and other dairy products so as to avoid exhibiting these undesirable symptoms. Several other approaches have also been developed to aid individuals with lactose intolerance, including creating lactose-free foods and the co-ingestion of lactase supplements with lactose-containing foods. Enzyme supplement treatments are particularly promising because they do not cause undesirable changes in food quality or nutritional profile. Typically, a tablet or capsule containing lactase is taken at the same time as a lactose-containing food, which promotes lactose hydrolysis within the gastrointestinal tract (GIT). β-gal is the most common form of lactase used in enzyme supplements, but it cannot simply be delivered in its free form because it is highly susceptible to denaturation and deactivation under GIT conditions. In particular, lactase is usually deactivated when exposed to the highly acidic gastric fluids present within the human stomach.

A pH-sensitive fluorescence probe (FRD) was therefore used to estimate the internal pH of the beads using a ratiometric fluorescence method. FRD has both pH-dependent (FITC) and pH-independent (TMR) fluorescence groups within its molecular structure. The confocal fluorescence microscopy measurements indicated that the emission intensity from the FITC channel (488 nm) decreased when the fluorescent dye was incubated in lower pH buffers, while the emission intensity from the TMR channel (543 nm) remained relatively constant (data not shown). A standard curve of fluorescence intensity ratio versus pH (from pH 4 to 7) was then obtained by taking the ratio of the emission intensities for the TMR/FITC channels (data not shown). The pH value inside the hydrogel beads could then be determined from the confocal fluorescence microscopy images using this calibration curve.

Confocal microscopy images of the hydrogel beads were taken before and after exposure to simulated gastric fluids (data not shown). For the buffer-free beads, the fluorescence intensity of the FITC channel (pH-dependent) changed from relatively strong before exposure to very weak after exposure, which is indicative of a substantial reduction in the internal pH of the beads based on the calibration curve (data not shown). The interior of the buffer-free beads was calculated to be around pH 6.9 before exposure, and below the limit of detection (pH<4) after exposure. These results suggest that the hydrogen ions (H⁺) from the acidic gastric fluids rapidly diffused into the hydrogel beads and decreased their internal pH. Conversely, for the buffer-loaded beads, the fluorescence intensity of the FITC channel only decreased slightly after incubation in simulated gastric fluids (data shot shown). Indeed, the interior of the buffer-loaded beads changed from around pH 7.2 before exposure to around pH 6.6 after exposure. This slight reduction in pH would not be expected to deactivate the enzyme. As mentioned earlier, this effect can be attributed to the fact that the encapsulated Mg(OH)₂ is insoluble under neutral conditions, but dissolves under acidic conditions. Consequently, when hydrogen ions from the gastric fluids diffuse into the hydrogel beads, some of the Mg(OH)₂ dissolves, which generates OH⁻ ions that neutralize the H⁺ ions, thereby maintaining a neutral pH inside the beads. Presumably, there was sufficient magnesium hydroxide initially present within the hydrogel beads to counteract all the hydrogen ions arising from the gastric fluids. These experiments demonstrate that the fluorescence dye used in this study is sensitive to pH changes, and that it can be used to measure the local pH inside the hydrogel beads.

The local pH that lactase experiences within the stomach phase influences its molecular conformation and enzyme activity. For this reason, the activity of lactase encapsulated in hydrogel beads containing different amounts of co-encapsulated Mg(OH)₂ was measured after incubation in simulated gastric fluids. The impact of bead dimensions on enzyme activity was determined by fabricating large and small hydrogel beads using either a simple syringe or a dedicated encapsulation unit, respectively. The hydrogel beads were then incubated in simulated gastric fluids for different times, and the enzyme activity was measured using a spectrophotometric method.

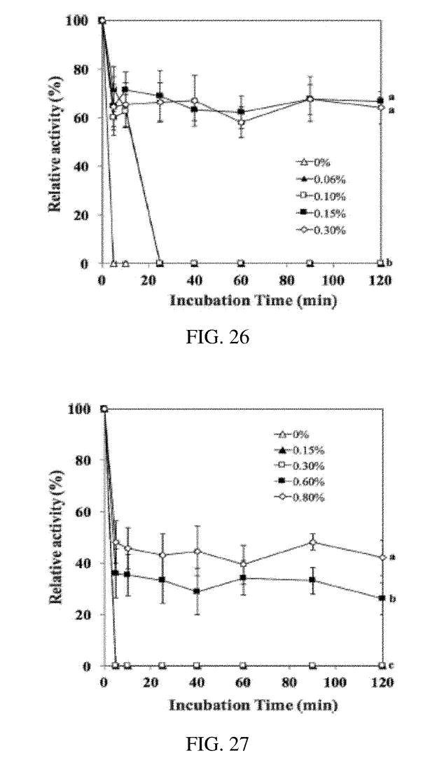

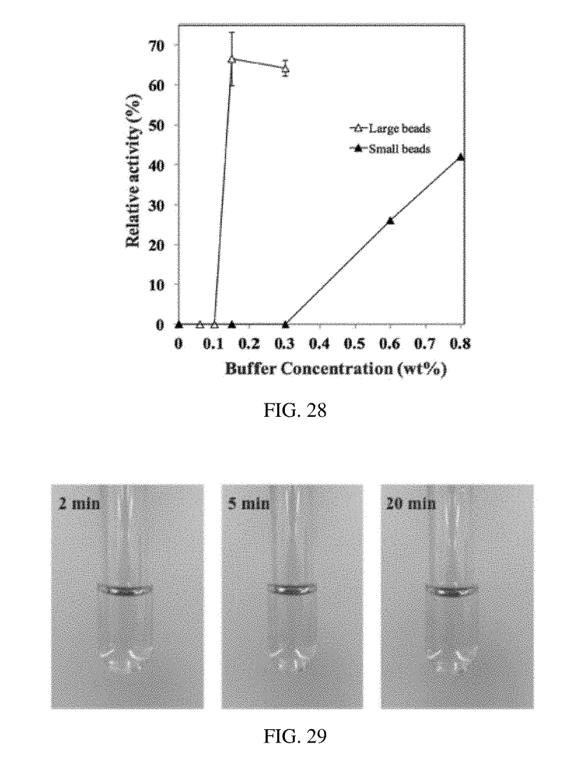

For both sizes of buffer-free beads, lactase activity was completely lost after 5 min incubation in simulated gastric fluids (FIGS. 26 and 27). Without being held to theory, it is believed that this type of hydrogel bead was unable to prevent acid-induced loss of lactase activity because small H⁺ ions easily diffuse through the pores in the biopolymer matrix and rapidly reduced the internal pH. The fact that bead dimensions did not have a major impact in this case was probably because the diffusion process occurred so quickly. Conversely, for the buffer-loaded beads, the activity of the encapsulated enzyme depended on both the level of Mg(OH)₂ present and the bead dimensions (FIGS. 26 and 27). Lactase activity decreased steeply after 5 minutes incubation for large buffer-loaded beads containing only 0.06% or 0.1% Mg(OH)₂, with a complete loss of enzyme activity after 25 min incubation (FIG. 26). These results suggest that there was insufficient buffer present inside the beads to completely neutralize all the hydrogen ions (H⁺) that diffused into them. At 0.15% and 0.3% Mg(OH)₂, the enzyme activity within the large buffer-loaded beads remained >60% after 120 min incubation (FIG. 28), which indicated that these systems were effective at preventing the acid-denaturation of lactase. This effect can be attributed to their ability to maintain a relatively high pH inside the hydrogel beads. For the small buffer-loaded beads, a higher amount (0.6 or 0.8%) of Mg(OH)₂ was needed to maintain a reasonable level of enzyme activity at the end of the gastric phase (26 or 42% respectively) (FIGS. 27 and 28). The observed impact of hydrogel bead dimensions on the activity of the enzyme can be attributed to the shorter distance that the H⁺ ions have to diffuse into smaller beads. In addition, the fraction of lactase molecules in close proximity to the surfaces of the hydrogel beads (and therefore more susceptible to acid denaturation) increases with decreasing bead diameter. This phenomenon may account for the fact that a higher enzyme activity was maintained at the end of the digestion period for the larger beads than the smaller ones (FIG. 28).

In the presence of sufficiently high levels of magnesium hydroxide the enzyme activity remained relatively high and constant after the first 5 minutes of incubation (FIGS. 26 and 27). This suggests that a fraction of the lactase was rapidly inactivated, while the rest remained in an active state. Without being held to theory, it is believed that the lactase molecules closest to the surface of the hydrogel beads were inactivated when hydrogen ions diffused into them and reduced the local pH before there was time for the magnesium hydroxide particles to dissolve and release neutralizing hydroxyl ions. In addition, some of the lactase molecules at the surfaces of the beads may have diffused into the surrounding gastric fluids and been inactivated.

Additional insights into the impact of encapsulation on the stability of lactase was obtained by measuring changes in the appearance of hydrogel bead suspensions incubated at pH 2.5 (gastric) and pH 7.0 (intestinal) in the presence of a lactase substrate (o-NPG). Photographic images of the samples were taken periodically to indicate color changes during incubation: an increase in the intensity of the yellow color of the samples is an indication of a higher conversion of o-NPG to galactose o-NP by lactase.

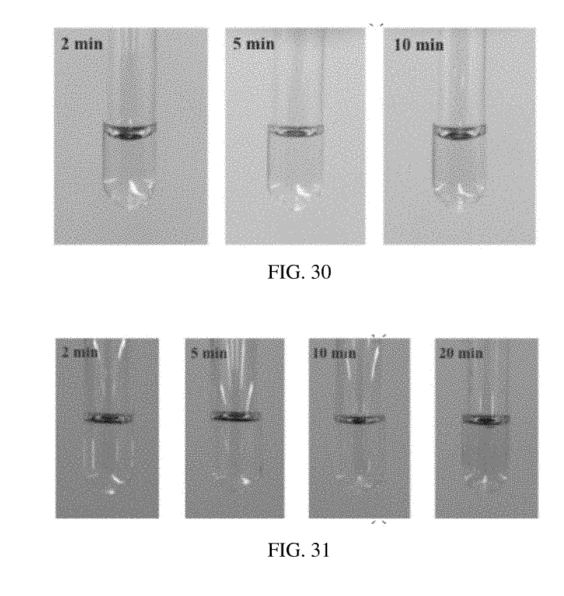

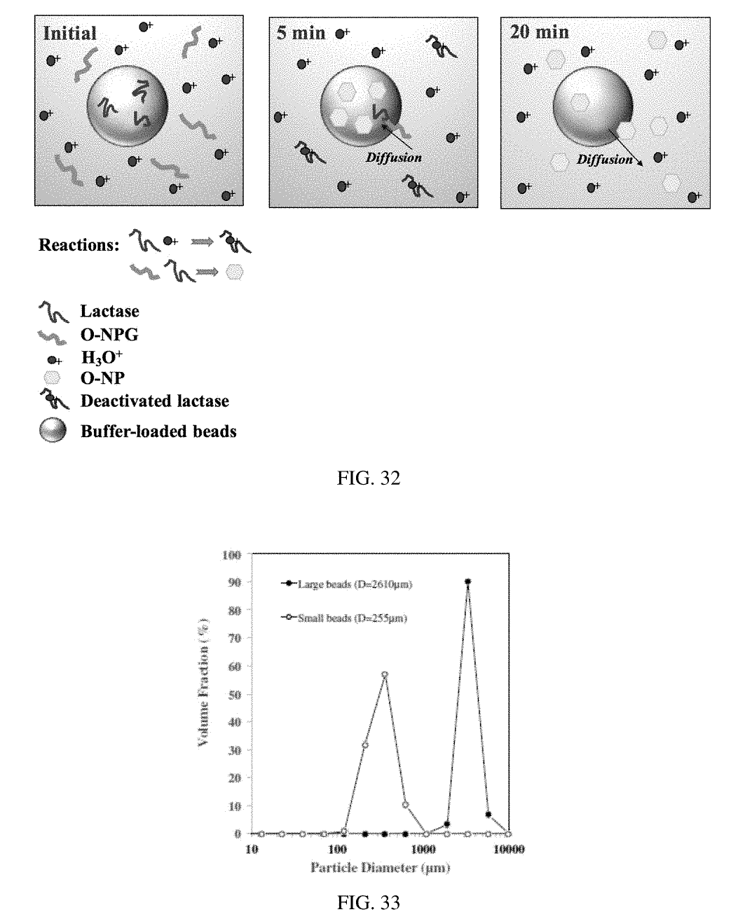

For the buffer-free beads, the yellow color was evenly distributed within the o-NPG solution during incubation at pH 7 (FIG. 30), which suggests a high enzyme activity under these conditions. Conversely, when buffer-free beads were incubated in the o-NPG solution at pH 2.5, no color change was observed indicating that the enzyme was completely deactivated under these acidic conditions (FIG. 29). For the buffer-loaded beads incubated at pH 2.5, the yellowish color first appeared in the hydrogel beads located at the bottom of the tube, then the external o-NPG solution became yellowish at longer incubation times (FIG. 31). Presumably, the substrate (o-NPG) initially diffused into the hydrogel beads where it was hydrolyzed by the encapsulated lactase. At longer times, some of the reaction products diffused out of the hydrogel beads and into the surrounding aqueous phase, thereby turning the whole solution yellow. A schematic diagram of the proposed reactions taking place in these systems is shown in FIG. 32.

The experimental studies indicated that the hydrogel bead dimensions had a major impact on the stability of the encapsulated lactase to acidic environments, which was attributed to the impact of particle size on the kinetics of H⁺ ion diffusion into the beads. In this section, the impact of bead dimensions on hydrogen ion diffusion were therefore investigated using a mathematical model.

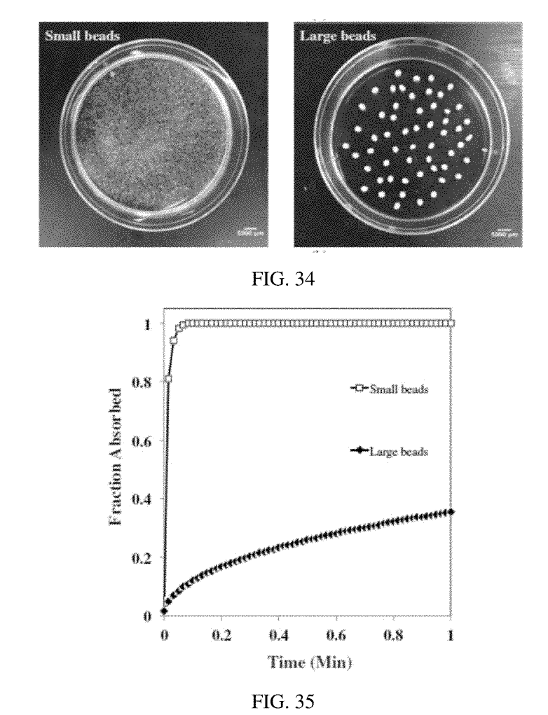

The particle size distributions of suspensions of both small and large hydrogel beads contained a single peak (FIG. 33). Digital photographs of the samples showed that they contained fairly uniform size particles that were evenly distributed throughout the system (FIG. 34). The mean particle diameters of the small and large beads were around 255 and 2610 μm, respectively.

The diffusion of hydrogen ions into hydrogel beads with different dimensions can be predicted using Equation (1). Φ refers to the fraction of H⁺ ions that have diffused into the hydrogel beads at time t, M(t) and M(∞) are the concentrations of H⁺ ions inside the hydrogel beads at time t and at equilibrium, n is an integer, a is the hydrogel bead radius, and D_{gel} is the diffusion coefficient of the H⁺ ions through the hydrogel bead matrix, which can be estimated based on Equation (2). D_{w} is the diffusion coefficient of the H⁺ ions through pure water, r_{H} is the hydrodynamic radius of the H⁺ ions, r_{f} is the cross-sectional radius of the biopolymer chains (κ-carrageenan) in the hydrogel network, and ζ is the mesh pore diameter of the hydrogel network. The radius (r_{f}) of the carrageenan chain has been reported to be around 0.50 nm. Typical pore sizes of biopolymer hydrogels are reported to be in the range 5 to 500 nm, and a value of 15 nm was assumed for the purpose of this study. This value was selected because it is at the low end of the pore size range, and therefore provides an estimate of the maximum potential of the beads to limit diffusion. The hydration (r_{H}) radius of the H⁺ ions (actually H₃O⁺ ions) has been estimated to be around 0.1 nm. The diffusion coefficient of H⁺ ions in water can be obtained from: D_{w}=k_{B}T/6πηr_{H}, where k_{B} is Boltzmann's constant, T is absolute temperature, and η is the viscosity of the solvent. These equations can be used to estimate the time-dependence of the fraction of H⁺ ions diffusing into hydrogel beads with different sizes after incubation in the stomach phase (FIG. 35).

The calculations highlight the importance of hydrogel bead dimensions on the diffusion rate of H⁺ ions into the beads. The predictions show that H⁺ ions can diffuse into the small beads much quicker (in a few seconds) than into the large ones, which would account for the fact that a higher amount of Mg(OH)₂ has to be loaded into the smaller beads to maintain a neutral internal pH. These calculations partly account for the reason that the smaller beads are less effective at protecting the lactase from acidic conditions than the larger beads

In practice, smaller hydrogel beads are often preferred for applications in the food industry because they give products with more desirable optical, rheological, stability, and mouthfeel characteristics. This study has shown that smaller beads can still be used to protect lactase from acid-induced degradation, but that a higher level of Mg(OH)₂ must be incorporated into the beads. In addition, it may be possible to further enhance enzyme activity by using different types and amounts of biopolymers or cross-linking agents to control the hydrogel pore size and physical interactions.

### Conclusions for Example 6

In this study, lactase was encapsulated within potassium carrageenan-based hydrogel beads using the injection-gelation method. The acid-stability of the enzyme could be improved by co-encapsulating it with a basic buffering agent: Mg(OH)₂. This buffer is insoluble under neutral conditions, but dissolves when hydrogen ions diffuse into the hydrogel beads, thereby ensuring a neutral internal pH (as long as some buffer remains). A quantitative ratiometric fluorescence method proved to be highly effective at determining the local pH inside the beads before and after exposure to simulated gastric conditions. The interior of the buffer-free beads rapidly decreased from pH 6.9 to <4 when exposed to the stomach phase, which led to rapid deactivation of the encapsulated lactase. Conversely, the interior of the buffer-loaded beads remained relatively close to neutral (pH 6.6) even after 2 hours exposure to simulated gastric fluids, which helped to retain lactase activity. A higher amount of Mg(OH)₂ was required to protect lactase encapsulated in small beads than in large beads, which was attributed to faster diffusion of H⁺ ions into the smaller bead interiors. This study suggests that buffer-loaded hydrogel beads may be suitable food-grade delivery systems for acid-sensitive enzymes, such as lactase. However, in vivo animal and human studies are stilled needed to confirm that buffer-loaded beads can maintain lactase activity under real life conditions.

The novel hydrogel beads disclosed herein can be used as an effective delivery system for acid sensitive bioactive agents based on pH measurement and enzyme assay.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A composition comprising a hydrogel bead in an external matrix, the hydrogel bead comprising
an at least partially crosslinked gelling polymer, the hydrogel bead having encapsulated therein
a functional agent, and
a buffering agent having low water solubility,
wherein at least a portion of the buffering agent is in solid form in the hydrogel bead.

2. The composition of claim 1, wherein the external matrix comprises water, air, an organic solvent, or an oil.

3. The composition of claim 1, wherein the pH in the hydrogel bead is higher or lower than the pH of the external matrix.

4. The composition of claim 1, wherein the gelling polymer comprises a polysaccharide, a glycoprotein, a glycopeptide, a protein carbohydrate conjugate, a protein lipid conjugate, a carbohydrate-lipid conjugate, a protein, or a combination comprising at least one of the foregoing.

5. The composition of claim 4, wherein the gelling polymer comprises alginate, carrageenan, agar, pectin, xanthan gum, chitosan, whey protein, caseinate, soy protein, pea protein, legume protein, gelatin, lactoferrin, or a combination comprising at least one of the foregoing.

6. The composition of claim 1, wherein the buffering agent having low water solubility comprises Ca(OH)₂, CaCO₃, Ca₃(PO₄)₂, Mg(OH)₂, MgCO₃, Zn(OH)₂, ZnCO₃, Zn₃(PO₄)₂, benzoic acid, or a combination comprising at least one of the foregoing.

7. The composition of claim 1, wherein the functional agent comprises a bioactive agent.

8. The composition of claim 7, wherein the bioactive agent comprises a protein, an enzyme, a nutraceutical, a probiotic, a prebiotic, a drug, or a combination comprising at least one of the foregoing.

9. The composition of claim 8, wherein the bioactive agent comprises β-galactosidase, lipase, lactase, phospholipase, amylase, pepsin, chymotrypsin, trypsin, a carotenoids (a-carotene, b-carotene, lycopene, lutein, and fucoxanthin), curcumin, resveratrol, a polyphenol, a phytosterol, a phytostannol, a flavonoid, Coenzyme Q10, or a combination comprising at least one of the foregoing.

10. The composition of claim 1, wherein the functional agent comprises a colorant, a flavoring, a nutrient, a supplement, or a combination comprising one or more of the foregoing.

11. The composition of claim 1, wherein the hydrogel beads have an average diameter of 500 nm to 5000 μm.

12. The composition of claim 7 in the form of an oral pharmaceutical or nutraceutical composition comprising a pharmaceutically acceptable carrier.

13. A method of orally administering a bioactive agent, comprising orally administering to a subject the composition of claim 12.

14. The method of claim 13, wherein upon orally administering, the buffering agent having low water solubility maintains a basic pH in the hydrogel beads through the subject's stomach.

15. The composition of claim 10 in the form of a food composition comprising a basal food composition.

16. The composition of claim 15, wherein upon ingestion, the buffering agent having low water solubility maintains a basic pH in the hydrogel beads through the subject's stomach

17. A method of making an encapsulated functional agent, comprising
mixing a gelling polymer, a functional agent, and a buffering agent having low water solubility to form a mixture,
forming hydrogel beads from the mixture, and
crosslinking at least a portion of the gelling polymer in the hydrogel beads to provide the encapsulated functional agent,
wherein at least a portion of the buffering agent is in solid form in the hydrogel bead.

18. The method of claim 17, wherein forming hydrogel beads from the mixture comprises passing the mixture through a vibrating nozzle.

19. The method of claim 17, wherein crosslinking at least a portion of the gelling polymer comprises incubating the hydrogel beads in a solution comprising a cationic crosslinking agent for a time sufficient to crosslink at least a portion of the gelling polymer.

20. The method of claim 19, wherein the cationic crosslinking agent comprises Ca²⁺, K⁺, Na⁺, chitosan, polylysine, or a combination comprising at least one of the foregoing.

21. The method of claim 17, wherein the gelling polymer comprises a polysaccharide, a glycoprotein, a glycopeptide, a protein carbohydrate conjugate, a protein lipid conjugate, a carbohydrate-lipid conjugate, a protein, or a combination comprising at least one of the foregoing.

22. The method of claim 17, wherein the buffering agent having low water solubility comprises one of Ca(OH)₂, CaCO₃, Ca₃(PO₄)₂, Mg(OH)₂, MgCO₃, Zn(OH)₂, ZnCO₃, Zn₃(PO₄)₂, benzoic acid, or a combination comprising at least one of the foregoing.

23. The method of claim 17, wherein the functional agent comprises a bioactive agent.

24. The method of claim 23, wherein the bioactive agent comprises a protein, an enzyme, a nutraceutical, a probiotic, a prebiotic, a drug, or a combination comprising at least one of the foregoing.

25. The method of claim 17, wherein the functional agent comprises a colorant, a flavoring, a nutrient, a supplement, or a combination comprising one or more of the foregoing.

26. The method of claim 17, wherein the hydrogel beads have an average diameter of 500 nm to 5000 μm.
